(19) United States
(12) Patent Application Publication
(10) Pub. No.: US 2026/0201028 A1
(43) Pub. Date: Jul. 16, 2026

(54) AN ANTI-TRPM4 ANTIBODY FOR TREATING TRAUMATIC BRAIN INJURY AND VASCULAR DEMENTIA

(71) Applicants: SINGAPORE HEALTH SERVICES PTE LTD, Singapore (SG); UNIVERSITI SAINS MALAYSIA, Penang (MY)

(72) Inventors: Ping LIAO, Singapore (SG); Zurina HASSAN, Penang (MY)

(73) Assignees: SINGAPORE HEALTH SERVICES PTE LTD, Singapore (SG); UNIVERSITI SAINS MALAYSIA, Penang (MY)

(21) Appl. No.: 19/137,997

(22) PCT Filed: Dec. 11, 2023
(86) PCT No.: PCT/SG2023/050821

§ 371 Date: Jun. 11, 2025

(30) Foreign Application Priority Data
Dec. 12, 2022 (SG) 10202260418Y

(51) Int. Cl. C07K 16/28; A61K 31/047; A61K 39/00; A61K 45/06; A61P 25/00; A61P 25/28

(52) U.S. Cl. CPC C07K 16/28; A61K 31/047; A61K 45/06; A61P 25/00; A61P 25/28; A61K 2039/505; C07K 2317/24; C07K 2317/565; C07K 2317/76; C07K 2317/92

(57) ABSTRACT

Disclosed are anti-transient receptor potential melastatin 4 (TRPM4) antibodies and their use for treating traumatic brain injury and vascular dementia. In particular, disclosed are humanized monoclonal antibodies specific to TRPM4 and their use for treating traumatic brain injury and vascular dementia.

### CROSS-REFERENCE TO RELATED APPLICATION

This application claims the benefit of priority of Singapore Application No. 10202260418Y, filed 12 Dec. 2022, the contents of it being hereby incorporated by reference in its entirety for all purposes.

### FIELD OF INVENTION

The present disclosure relates to anti-transient receptor potential melastatin 4 (TRPM4) antibodies and their use for treating traumatic brain injury and vascular dementia. In particular, the present disclosure relates to humanized monoclonal antibodies specific to TRPM4 and their use for treating traumatic brain injury and vascular dementia.

### BACKGROUND

TRPM4 is a nonselective cation channel, permeable to monovalent cation channels. Once upregulated and activated under hypoxia or stress conditions, excessive Na⁺ entry via TRPM4 channel could lead to oncotic cell death. Acute and/or chronic hypoxia are present after TBI or during the progress of vascular dementia. TRPM4 plays a similar role in vascular and neuro injury during this process. Therefore, blocking TRPM4 enables protection of vasculature and improvement of neural protection.

### Traumatic Brain Injury

Traumatic brain injury (TBI) is an injury to the brain parenchyma caused by an external force and is usually prevalent in young to middle aged adults with profound socioeconomic consequences from associated disabilities. Annually, approximately 27-69.0 million people are diagnosed with TBIs, with an incidence of 351-939 cases per 100,000 population. Motor vehicle accidents and falls are the two most common causes. Morbidity and mortality of TBI are high, particularly in young adults <45 years of age.

Traumatic brain injury has a very complex, dynamic and heterogeneous pathology, leading to a wide variety of injuries. Initially, direct tissue damage (known as the primary injury) occurs, leading to immediate (usually in milliseconds to seconds), irreversible brain damage such as hemorrhage, tissue destruction and axonal shearing. Within hours to days, the primary injury induces a secondary brain damage on the cellular, metabolic and molecular level such as oxidative stress, inflammation and apoptosis. The development of edema and subsequent cerebral hypertension during this phase becomes a major factor for the high mortality and morbidity and is the leading cause of death in more than half of all deaths after TBI. At least 80% of severe TBI patients have elevated intracranial pressure (ICP) and ICP monitoring is recommended by clinical practice guidelines. Intracranial hypertension is dangerous as the skull is unable to expand, and therefore, the pressure can damage the brain or spinal cord by pressing on important brain structures such as brain stem and by restricting blood flow into the brain.

The mainstay of treatment of TBI to prevent secondary injury is surgical evacuation of hematoma and mass lesions (brain contusion), cerebrospinal fluid (CSF) removal and bony decompression. This is followed by multimodality monitoring and neurocritical care. The ultimate aim is to maintain cerebral perfusion and reduce intracranial pressure.

To ameliorate the severity of secondary injuries such as edema and hemorrhage, pharmacological approaches are required, which will benefit TBI patients and improve their functional outcome. During the development of edema and hemorrhage secondary to TBI, blood-brain barrier (BBB) disruption plays a central role. BBB is mainly formed by the tight-junctions of the endothelium of the cerebral blood vessels. After TBI, hypoxic conditions within certain damaged areas and cytotoxic substances released from dead tissues are known contributors to BBB derangement. With the loss of BBB integrity, fluid from circulation will flux into brain parenchyma, contributing to vasogenic edema. In the worst-case scenario, secondary bleeding may occur, worsening the increase of intracranial pressure.

### Vascular Dementia

Chronic hypoxia is associated with vascular dementia, and chronic excitotoxicity has been postulated to contribute to the progression of vascular dementia. One option to control excitotoxicity in brain disorders with chronic hypoxia is by blocking TRPM4.

Vascular dementia is a common type of dementia caused by reduced blood flow to the brain. Conditions such as atherosclerosis and obesity interrupt the flow of blood and oxygen supply to the brain and damage blood vessels in the brain. Cerebrovascular disease is likely the most common cause of cognitive impairment even above Alzheimer's disease (AD). During the development of vascular dementia, injuries to both small vessels and large vessels in the brain could lead to cognitive decline.

In elderly, cerebral small vessel disease is the most common, chronic and progressive vascular disease affecting arterioles, capillaries and small veins. Damages to the brain can be diagnosed by Magnetic Resonance Imaging (MRI). Small vessel disease can be found in around 45% of dementia patients. Common causes include arteriosclerosis, cerebral amyloid angiopathy (CAA), genetic small vessel angiopathy, inflammation and immune-mediated small vessel diseases, and venous collagenosis.

Narrowing of the large vessels to the brain also impairs cognition. Extracranial carotid stenosis (ECS) is a narrowing of the lumen of the carotid artery, usually caused by atherosclerosis. As the third leading cause of ischemic stroke in the general population, ECS is associated with stroke via several mechanisms: atheroembolism of cholesterol crystals or other debris, artery to artery embolism of thrombus, structural disintegration of the wall (dissection), acute thrombotic occlusion, and reduced cerebral perfusion with plaque growth. Approximately 7-18% of all first stroke cases are associated with ECS. Based on the Framingham Heart Study and Cardiovascular Health Study populations, the prevalence of severe carotid stenosis (>50% narrowing) is approximately 9% in men and 6-7% in women.

Some ECS patients may experience warning symptoms of a stroke named transient ischemic attack (TIA), which requires a medical emergency. Other symptoms of ECS may overlap with vertebral artery disease including dizziness, vertigo, double vision, numbness around the mouth, tinnitus (ringing in the ears), difficulty speaking, and partial blindness. However, many patients do not exhibit any symptom. In symptomatic patients, there is a clear correlation between the degree of stenosis and the risk of stroke. Therefore, surgical interventions such as carotid endarterectomy (CEA) or carotid angioplasty and stenting (CAS) are selected to treat symptomatic ECS patients, in particular in high-grade (70%-99%) stenosis. The benefit is rather modest for patients with less severe stenosis (50%-69%). Carotid revascularization is not recommended for patients with stenosis <50% since the risk for stroke is low in these patients.

For asymptomatic ECS patients, doctors have not reached a consensus on the use of surgical interventions to recanalize carotid artery. First, the benefits of CEA or CAS for stroke risk reduction in asymptomatic patients are less profound compared to symptomatic patients. Second, the inherent risk for perioperational complications, such as stroke and myocardial infarction, remains a concern for safety and efficacy. Therefore, currently, the only choice for asymptomatic patients is medical management including providing antithrombotic therapy, providing treatment of hypertension, hyperlipidemia, and diabetes mellitus, controlling obesity and encouraging smoking cessation.

Cognitive impairment is common in both symptomatic and asymptomatic ECS patients. In high-grade stenosis, cognitive decline is often associated with silent embolization, but it may also develop without evidence of infarction. For example, the Tromsø Study and the Cardiovascular Health Study demonstrated that cognitive impairment in patients with carotid stenosis was independent of vascular lesions observed on MRI. The efficacy of surgical intervention on cognitive impairment is also unclear. On one hand, carotid artery recanalization may improve cognition by enhancing cerebral perfusion. On the other hand, complications may arise from operation that damage cognition. For example, microembolization may develop after carotid interventions. In asymptomatic ECS patients, the efficacy of carotid revascularization on cognition is even less prominent. In the Asymptomatic Carotid Atherosclerosis Study, CEA was shown to have no effect on cognitive functional improvement.

As current non-surgical treatments for ECS mainly focus on disease prevention such as controlling hypertension and diabetes, no specific treatment for neuroprotection is available, in particular for managing cognitive impairment.

In view of the above-mentioned challenges in TBI and vascular dementia, there is a need for a novel way to manage vascular and neural protection for TBI and vascular dementia. In particular, there is a need to develop new molecules (such as antibodies) and methods to manage TBI and vascular dementia.

### SUMMARY

In one aspect, the present disclosure refers to a method of treating traumatic brain injury (TBI), comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one aspect, the present disclosure refers to use of an effective amount of a humanized monoclonal antibody or antigen-binding fragment thereof specific to a transient receptor potential melastatin 4 (TRPM4) protein in the manufacture of a medicament for treating TBI, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one aspect, the present disclosure refers to a method of treating vascular dementia, comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one aspect, the present disclosure refers to use of an effective amount of a humanized monoclonal antibody or antigen-binding fragment thereof specific to a transient receptor potential melastatin 4 (TRPM4) protein in the manufacture of a medicament for treating vascular dementia, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

### BRIEF DESCRIPTION OF DRAWINGS

The present disclosure will be better understood with reference to the detailed description when considered in conjunction with the non-limiting examples and the accompanying drawings, in which:

FIG. 1 (comprised of FIGS. 1A, 1B, 1C, 1D, 1E, and 1F) shows that blocking TRPM4 improved functional recovery following TBL FIG. 1A shows the immunostaining of neuronal marker NeuN and TRPM4 one day after the induction of TBI in animals (rats). FIG. 1B shows the mRNA expression of TRPM4 one day after TBI induction from real time PCR. FIG. 1C shows the number of total footslips recorded during beam walking test in animals (rats) with skull fracture following TBI induction. M4P or IgG was given at a dose of 0.4 mg/kg body weight right after TBI induction. Sham operation was performed as control. FIG. 1D illustrates the results of tail suspension assay, showing immobility time in rats with skull fracture. FIG. 1E shows the results of beam walking test in rats without skull fracture following TBI induction. FIG. 1F shows the results of tail suspension test in rats without skull fracture following TBI induction. * p<0.05, ** p<0.01, *** p<0.001, #p<0.0001, vs sham.

FIG. 2 (comprised of FIGS. 2A, 2B, 2C, 2D, 2E, 2F, 2G, 2H, 2I, 2J, 2K, 2L, 2M, 2N, and 2O) demonstrates that TRPM4 blocking antibody attenuated excitotoxicity in neurons under chronic hypoxia. FIG. 2A is a western blot showing the expression of TRPM4 in cultured hippocampal neurons under normoxia or 24 hr chronic hypoxia. FIG. 2B shows the summary of TRPM4 protein expression. FIG. 2C shows the change of intracellular calcium concentration ([Ca²⁺]ᵢ) in normoxic and hypoxic neurons upon 2 μM glutamate stimulation. FIG. 2D shows the change of [Ca²⁺]ᵢ in normoxic and hypoxic neurons upon 100 μM NMDA stimulation. FIG. 2E shows the resting membrane potential (RMP) of a normoxic neuron and a neuron exposed to hypoxia for 24 hr. RMPs were obtained using I=0 current-clamp mode when neurons were broken in as whole-cell configuration. FIG. 2F shows the current-voltage relationship for hippocampal neurons under normoxia (n=9 cells) and 24 hr hypoxia (n=9 cells). A 250-ms voltage ramp protocol was applied from −70 mV holding membrane potential. FIG. 2G shows the current-voltage relationship for hippocampal neurons under normoxia with the treatment of control IgG and M4P. FIG. 2H shows the current-voltage relationship for hippocampal neurons under hypoxia with the treatment of IgG and M4P. FIG. 2I shows the sample traces of RMP from hypoxic hippocampal neurons receiving IgG or M4P treatment. FIG. 2J shows the summary of cell capacitance for hypoxic neurons under IgG and M4P treatments. n=10 cells. FIG. 2K shows the kinetic of [Ca²⁺]ᵢ change in response to 2 μM glutamate stimulation under normoxia. FIG. 2L shows the changes of [Ca2+]i under chronic hypoxia. FIG. 2M shows the kinetic of [Ca²⁺]ᵢ change in response to 10 μM NMDA stimulation under normoxia. FIG. 2N shows the changes of [Ca²⁺]ᵢ in response to 100 μM NMDA under chronic hypoxia. FIG. 2O shows the protein expression of apoptotic marker Cleaved Caspase 3 in chronic hypoxic neurons treated with IgG or M4P * p<0.05, ** p<0.01. Student's t test.

FIG. 3 (comprised of FIGS. 3A, 3B, 3C, 3D and 3E) shows that blocking TRPM4 reduced apoptosis and improved cognition in animal model of bilateral common carotid artery occlusion (BCCAO). FIG. 3A shows the protein expression of Cleaved Caspase-3 in hippocampal neurons of BCCAO following treatment of IgG or M4P. FIG. 3B is a western blot for Cleaved Caspase-3 in hippocampus of BCCAO rats. FIG. 3C shows the amplitude of field excitatory postsynaptic potential (fEPSP) after theta-burst stimulation (TBS) of the Schaffer collaterals. N=6. FIG. 3D shows the summary of percentage of time spent in target quadrant within 60 s in the probe trial, in the Morris water maze experiment. n=6. FIG. 3E shows the escape latency in the BCCAO rats to locate the hidden platform during 5-days training 1 month after BCCAO. n=6. *P<0.05, ** P<0.01, ***P<0.001 for M4P vs IgG.

FIG. 4 (comprised of FIGS. 4A, 4B and 4C) illustrates the characterization of humanized anti-TRPM4 antibodies. FIG. 4A shows the immunofluorescent staining of humanized antibodies in HEK 293 cells transfected with human TRPM4. Human IgG was transfected in HEK 293 as control. Antibody clone A1 is an anti-TRPM4 chimeric antibody. Antibody clones A2-A7 are humanized anti-TRPM4 antibodies. Myc staining showed successful transfection of human TRPM4 and Wheat germ agglutinin (WGA) was used as surface glycoproteins staining. FIG. 4B is a bar graph showing the electrophysiological characterization of the chimeric antibody clone (A1) and the humanized antibody clones (A2-A7). TRPM4 currents at 0 min and 7 min hypoxia were recorded. Currents at +80 mV and −80 mV were summarized in the bar graph. FIG. 4C is a dose-response curve showing dose-dependent inhibition of humanized antibody clone, A2 on hypoxia-induced current increase. IC₅₀:1.23 μg/ml.

FIG. 5 are sensor-gram plots showing the binding affinity and kinetics of different humanized antibodies to Peptide-biotin. The overlaid smooth black lines represent the kinetic fit of the binding response signals at different antibody concentrations to a 1:1 interaction model.

FIG. 6 (comprised of FIGS. 6A, 6B and 6C) are dynamic light scattering (DLS) thermograms of humanized antibody clones A2 (VH1+VL1), A4 (VH1+VL3) and A6 (VH2+VL1) under different temperatures. FIG. 6A shows that humanized antibody clone A2 (VH1+VL1) was stable at high temperature above 60° C. FIG. 6B shows that humanized antibody clone A4 (VH1+VL3) was stable at high temperature above 60° C. FIG. 6C shows that humanized antibody clone A6 (VH2+VL1) was stable at high temperature above 60° C.

FIG. 7 (comprised of FIGS. 7A, 7B and 7C) are capillary electrophoresis-sodium dodecyl sulfate-non-reduced (CE-SDS-NR) electropherograms of humanized antibody clones A2 (VH1+VL1), A4 (VH1+VL3) and A6 (VH2+VL1) being incubated at 40° C. for 28 days. DO (day 0) indicates the starting point. FIG. 7A shows that humanized antibody clone A2 (VH1+VL1) was stable at 40° C. for 28 days. FIG. 7B shows that humanized antibody clone A4 (VH1+VL3) was stable at 40° C. for 28 days. FIG. 7C shows that humanized antibody clone A6 (VH2+VL1) was stable at 40° C. for 28 days.

FIG. 8 (comprised of FIGS. 8A, 8B and 8C) are CE-SDS-NR electropherograms of humanized antibody clones A2 (VH1+VL1), A4 (VH1+VL3) and A6 (VH2+VL1) which shows the stability of the antibodies under low pH3.5 for 0, 2 and 4 hours. FIG. 8A shows that humanized antibody clone A2 (VH1+VL1) was stable at low pH 3.5 for 4 hours. FIG. 8B shows that humanized antibody clone A4 (VH1+VL3) was stable at low pH 3.5 for 4 hours. FIG. 8C shows that humanized antibody clone A6 (VH2+VL1) was stable at low pH 3.5 for 4 hours.

### DETAILED DESCRIPTION

The present disclosure describes humanized monoclonal antibodies specific to TRPM4 and their use for treating traumatic brain injury (TBI) and vascular dementia. The use of humanized antibodies specific to human TRPM4 is expected to demonstrate enhanced therapeutic effects especially in human patients due to lower risk of immune rejection compared to using rabbit or mouse antibodies known in the art.

In one example, the present disclosure refers to a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one example, the present disclosure refers to a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO:10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example, each heavy and light chain comprises one variable region and one constant region. It is known in the art that the variable region of the heavy and light chain region of an antibody is for antigen binding. In one example, the variable region comprises three complementarity-determining regions (i.e., CDR1, CDR2 and CDR3) and four framework regions (i.e., FR1, FR2, FR3 and FR4). In one example, the complementarity-determining regions and framework regions are in the order of FR1-CDR1-FR2-CDR2-FR3-CDR3-FR4.

In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 13. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 14. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A2. In one example, an antibody or antigen-binding fragment thereof having a heavy chain variable region comprising an amino acid sequence of SEQ ID NO: 7, and a light chain variable region comprising an amino acid sequence of SEQ ID NO: 11 corresponds to humanized antibody clone A2. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A3. In one example, an antibody or antigen-binding fragment thereof having a heavy chain variable region comprising an amino acid sequence of SEQ ID NO: 7, and a light chain variable region comprising an amino acid sequence of SEQ ID NO: 12 corresponds to humanized antibody clone A3. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13 corresponds to humanized antibody clone A4. In one example, an antibody or antigen-binding fragment thereof having a heavy chain variable region comprising an amino acid sequence of SEQ ID NO: 7, and a light chain variable region comprising an amino acid sequence of SEQ ID NO: 13 corresponds to humanized antibody clone A4. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14 corresponds to humanized antibody clone A5. In one example, an antibody or antigen-binding fragment thereof having a heavy chain variable region comprising an amino acid sequence of SEQ ID NO: 7, and a light chain variable region comprising an amino acid sequence of SEQ ID NO: 14 corresponds to humanized antibody clone A5. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6. In one example, an antibody or antigen-binding fragment thereof having a heavy chain variable region comprising an amino acid sequence of SEQ ID NO: 8, and a light chain variable region comprising an amino acid sequence of SEQ ID NO: 11 corresponds to humanized antibody clone A6. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A7. In one example, an antibody or antigen-binding fragment thereof having a heavy chain variable region comprising an amino acid sequence of SEQ ID NO: 8, and a light chain variable region comprising an amino acid sequence of SEQ ID NO: 12 corresponds to humanized antibody clone A7.

In one example, the present disclosure refers to an antibody or antigen-binding fragment thereof disclosed herein, wherein the antibody or antigen-binding fragment thereof comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO. 55, or SEQ ID NO: 56.

In one example, the present disclosure refers to an antibody or antigen-binding fragment thereof disclosed herein, wherein the antibody or antigen-binding fragment thereof comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 59. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 60. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 58.

In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A2. In one example, an antibody or antigen-binding fragment thereof having a heavy chain comprising an amino acid sequence of SEQ ID NO: 50, and a light chain comprising an amino acid sequence of SEQ ID NO: 57 corresponds to humanized antibody clone A2. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A3. In one example, an antibody or antigen-binding fragment thereof having a heavy chain comprising an amino acid sequence of SEQ ID NO: 50, and a light chain comprising an amino acid sequence of SEQ ID NO: 58 corresponds to humanized antibody clone A3. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59 corresponds to humanized antibody clone A4. In one example, an antibody or antigen-binding fragment thereof having a heavy chain comprising an amino acid sequence of SEQ ID NO: 50, and a light chain comprising an amino acid sequence of SEQ ID NO: 59 corresponds to humanized antibody clone A4. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60 corresponds to humanized antibody clone A5. In one example, an antibody or antigen-binding fragment thereof having a heavy chain comprising an amino acid sequence of SEQ ID NO: 50, and a light chain comprising an amino acid sequence of SEQ ID NO: 60 corresponds to humanized antibody clone A5. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6. In one example, an antibody or antigen-binding fragment thereof having a heavy chain comprising an amino acid sequence of SEQ ID NO: 51, and a light chain comprising an amino acid sequence of SEQ ID NO: 57 corresponds to humanized antibody clone A6. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A7. In one example, an antibody or antigen-binding fragment thereof having a heavy chain comprising an amino acid sequence of SEQ ID NO: 51, and a light chain comprising an amino acid sequence of SEQ ID NO: 58 corresponds to humanized antibody clone A7.

It is known in the art that the constant region of the heavy and light chain region of an antibody comprises a more conserved amino acid sequence compared to the variable region. In one example, the antibody or antigen-binding fragment disclosed herein comprises a constant region selected from the major classes of immunoglobulins, such as IgG, IgD, IgE, IgA and IgM. In one example, the constant region is selected from the group consisting of IgG1, IgG2, IgG3, and IgG4. In one example, the constant region is IgG1. In another example, the constant region is IgG4. In one example, the antibody or antigen-binding fragment thereof disclosed herein comprises a heavy chain comprising an IgG1 constant region, where said heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO; 50, SEQ ID NO: 51, SEQ ID NO: 52, SEQ ID NO: 53, SEQ ID NO: 54 or SEQ ID NO: 55. In one example, the antibody or antigen-binding fragment thereof disclosed herein comprises a heavy chain comprising an IgG4 constant region, where said heavy chain comprises an amino acid sequence having at least 85%, 86%, 87%, 88%, 89%, 90%, 91%, 92%, 93%, 94%, 95%, 96%, 97%, 98%, or 99% sequence identity SEQ ID NO: 56.

In one example, the present disclosure refers to an antibody or antigen-binding fragment thereof disclosed herein, wherein the antibody specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

In one example, the antibody or antigen-binding fragment thereof disclosed herein inhibits TRPM4 activity. In one example, the antibody or antigen-binding fragment thereof disclosed herein inhibits TRPM4 activity by inhibiting TRPM4 current. In one example, the antibody or antigen-binding fragment thereof disclosed herein inhibits TRPM4 activity by internalizing membrane TRPM4 protein. In one example, the antibody or antigen-binding fragment thereof disclosed herein inhibits TRPM4 activity by inhibiting TRPM4 current and internalizing membrane TRPM4 protein. In one example, under diseased conditions, inhibiting TRPM4 activity refers to blocking, preventing or disrupting the upregulated or activated protein function of TRPM4.

In one example, the concentration of the antibody or antigen-binding fragment thereof required to inhibit TRPM4 activity in vitro is from 1 μg/ml to 5 μg/ml, or from 2 μg/ml to 4 μg/ml, or about 1 μg/ml, or about 2 μg/ml, or about 3 μg/ml, or about 4 μg/ml, or about 5 μg/ml. In one example, the concentration of the antibody or antigen-binding fragment thereof required to inhibit TRPM4 activity in vitro is 1.23 μg/ml. In one example, the concentration of the antibody or antigen-binding fragment thereof required to inhibit TRPM4 activity in vitro may be determined by a dose-dependent assay detecting for the dose-dependent effect of the antibody or antigen-binding fragment thereof on hypoxia-induced TRPM4 current increase.

In one example, the present disclosure refers to a nucleic acid encoding the antibody or antigen-binding fragment thereof disclosed herein. In one example, the nucleic acid comprises a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 61. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 62. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 63.

In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively corresponds to humanized antibody clone A2. In one example, an antibody or antigen-binding fragment thereof having its heavy chain and its light chain encoded by polynucleotide sequences of SEQ ID NOs: 17 and 21, respectively, corresponds to humanized antibody clone A2. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively corresponds to humanized antibody clone A3. In one example, an antibody or antigen-binding fragment thereof having its heavy chain and its light chain encoded by polynucleotide sequences of SEQ ID NOs: 17 and 22, respectively, corresponds to humanized antibody clone A3. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively corresponds to humanized antibody clone A4. In one example, an antibody or antigen-binding fragment thereof having its heavy chain and its light chain encoded by polynucleotide sequences of SEQ ID NOs: 17 and 23, respectively, corresponds to humanized antibody clone A4. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively corresponds to humanized antibody clone A5. In one example, an antibody or antigen-binding fragment thereof having its heavy chain and its light chain encoded by polynucleotide sequences of SEQ ID NOs: 17 and 24, respectively, corresponds to humanized antibody clone A5. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively corresponds to humanized antibody clone A6. In one example, an antibody or antigen-binding fragment thereof having its heavy chain and its light chain encoded by polynucleotide sequences of SEQ ID NOs: 18 and 21, respectively, corresponds to humanized antibody clone A6. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively corresponds to humanized antibody clone A7. In one example, an antibody or antigen-binding fragment thereof having its heavy chain and its light chain encoded by polynucleotide sequences of SEQ ID NOs: 18 and 22, respectively, corresponds to humanized antibody clone A7.

In one example, the present disclosure refers to an expression vector comprising the nucleic acid disclosed herein. Suitable expression vectors for production of recombinant proteins (such as antibodies, such as the humanized antibodies of the present disclosure) are well known to those skilled in the art and examples of expression vectors comprising the nucleic acid disclosed herein include pcDNA3.1, pGEX and pCMV.

In one example, the present disclosure refers to a host cell comprising the nucleic acid disclosed herein. In one example, the present disclosure refers to a host cell comprising the expression vector disclosed herein. In one example, the present disclosure refers to a host cell comprising the nucleic acid disclosed herein and the expression vector disclosed herein. In one example, the host cell comprising the nucleic acid disclosed herein and/or the expression vector disclosed herein is human embryonic kidney 293 cells (HEK 293 cells) or Chinese hamster ovary cells (CHO cells). In one example, the host cell comprising the nucleic acid disclosed herein and/or the expression vector disclosed herein may be cells generally used for the production of humanized antibodies which are known to those skilled in the art.

In one example, the present disclosure refers to a method of producing the antibody or antigen-binding fragment thereof disclosed herein. In one example, the method comprises culturing the host cell disclosed herein in a culture medium. In one example, the culture medium is Dulbecco's Modified Eagle's Medium (DMEM) (12800017; Thermo Fisher Scientific, USA) supplemented with 10% fetal bovine serum (10500064, Thermo Fisher Scientific, USA); 1.74 g/L sodium bicarbonate; 1.2 g/L HEPES; and 100 U/mL Penicillin-Streptomycin (15140122; Thermo Fisher Scientific, USA), or any other suitable cell culture medium known in the art. In one example, the method further comprises isolating the antibody or antigen-binding fragment thereof from the culture medium. In one example, the method of producing and isolating the antibody or antigen-binding fragment thereof disclosed herein may be methods generally used for the production of recombinant proteins, such as humanized antibodies, which are known to those skilled in the art.

In one example, point mutation(s) has been made to the antibody or antigen-binding fragment thereof disclosed herein to stabilize the resulting antibody clone. In one example, at least one point mutation(s) has been made in a framework region(s) of the antibody to stabilize the resulting antibody clone. In one example, one point mutation has been made in a framework region(s) of the antibody to stabilize the resulting antibody clone. In one example, two point mutations have been made in a framework region(s) of the antibody to stabilize the resulting antibody clone. In one example, three point mutations have been made in a framework region(s) of the antibody to stabilize the resulting antibody clone. In one example, the at least one point mutation(s) can be made in FR1, FR2, FR3 or FR4, or combinations thereof, of the framework region of the antibody. In one example, one point mutation has been made in FR2 of the framework region of the antibody. In one example, two point mutations have been made in FR2 of the framework region of the antibody. In one example, one point mutation has been made in FR3 of the framework region of the antibody. In one example, two point mutations have been made in FR3 of the framework region of the antibody. In one example, three point mutations have been made in FR3 of the framework region of the antibody. In one example, there is no upper limit in the number of mutations made to a framework region of the antibody. In one example, one or more point mutations can be made in any framework region of any heavy chain variable region (such as the heavy chain variable region comprising an amino acid having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10) or any light chain variable region (such as the light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14). In one example, the term “point mutation” refers to a mutation where a single amino acid is substituted, inserted or deleted from an amino acid sequence.

In another example, modification(s) can be made to a constant region of the heavy chain and/or light chain to reduce toxicity. In one example, the modification(s) is point mutation(s). In one example, point mutation(s) has been made to the constant region of the heavy chain of the antibody or antigen-binding fragment thereof disclosed herein to reduce toxicity. In one example, at least one point mutation(s) has been made in the constant region of the heavy chain (or heavy chain constant region) of the antibody to reduce toxicity. In one example, one point mutation has been made in the heavy chain constant region of the antibody to reduce toxicity. In one example, two point mutations have been made in the heavy chain constant region of the antibody to reduce toxicity. In one example, the at least one point mutation(s) can be made in the IgG1, IgG2, IgG3 or IgG4 heavy chain constant region of the antibody or antigen-binding fragment thereof disclosed herein to reduce toxicity. In one example, the at least one point mutation(s) can be made in the IgG1 heavy chain constant region of the antibody or antigen-binding fragment thereof disclosed herein to reduce toxicity. In one example, the at least one point mutation(s) can be made in the IgG4 heavy chain constant region of the antibody or antigen-binding fragment thereof disclosed herein to reduce toxicity. In one example, one point mutation has been made in the IgG1 heavy chain constant region of the antibody to reduce toxicity. In one example, two point mutations have been made in the IgG1 heavy chain constant region of the antibody to reduce toxicity. In one example, one point mutation has been made in the IgG4 heavy chain constant region of the antibody to reduce toxicity. In one example, there is no upper limit in the number of point mutations made to a heavy chain constant region of the antibody. In one example, the LL residues at positions 261 and 262 of IgG1 heavy chain constant region (e.g. positions 261 and 262 of IgG1 heavy chain constant region of VH5) are substituted with AA residues. In one example, substitution of LL residues at positions 261 and 262 of IgG1 heavy chain constant region (e.g. positions 261 and 262 of IgG1 heavy chain constant region of VH5) with AA residues results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54. In one example, the N residue at position 324 of IgG1 heavy chain constant region (e.g. position 324 of IgG1 heavy chain constant region of VH6) is substituted with an A residue. In one example, substitution of N residue at position 324 of IgG1 heavy chain constant region (e.g. position 324 of IgG1 heavy chain constant region of VH6) with an A residue results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55. In one example, the S residue at position 254 of IgG4 heavy chain constant region (e.g. position 254 of IgG4 heavy chain constant region of VH7) is substituted with a P residue. In one example, substitution of S residue at position 254 of IgG4 heavy chain constant region (e.g. position 254 of IgG4 heavy chain constant region of VH7) with a P residue results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56.

In one example, the present disclosure refers to a pharmaceutical composition comprising the antibody or antigen-binding fragment thereof disclosed herein, and a pharmaceutically acceptable carrier, excipient, or diluent. In one example, the present disclosure refers to the antibody or antigen-binding fragment thereof disclosed herein for use as a medicament.

In one example, the present disclosure refers to a method of treating traumatic brain injury, comprising administering to a subject an effective amount of the antibody or antigen-binding fragment thereof disclosed herein, or the pharmaceutical composition disclosed herein. Therefore, in one aspect, the present disclosure refers to a method of treating traumatic brain injury (TBI), comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6. In one example, the antibody or antigen-binding fragment thereof for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example of the method disclosed herein, each heavy and light chain of the antibody or antigen-binding fragment thereof comprises one variable region and one constant region. It is known in the art that the variable region of the heavy and light chain region of an antibody is for antigen binding. In one example of the method disclosed herein, the variable region comprises three complementarity-determining regions (i.e., CDR1, CDR2 and CDR3) and four framework regions (i.e., FR1, FR2, FR3 and FR4). In one example of the method disclosed herein, the complementarity-determining regions and framework regions are in the order of FR1-CDR1-FR2-CDR2-FR3-CDR3-FR4. The term “traumatic brain injury” (interchangeable with “traumatic head injury”) refers to a head injury or brain injury caused by physical event or external force.

In one example, the present disclosure refers to use of an effective amount of the antibody or antigen-binding fragment thereof disclosed herein, or the pharmaceutical composition disclosed herein, in the manufacture of a medicament for treating TBI. Therefore, in one aspect, the present disclosure refers to use of an effective amount of a humanized monoclonal antibody or antigen-binding fragment thereof specific to a transient receptor potential melastatin 4 (TRPM4) protein in the manufacture of a medicament for treating TBI, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6. In one example, the antibody or antigen-binding fragment thereof for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example of the use disclosed herein, each heavy and light chain of the antibody or antigen-binding fragment thereof comprises one variable region and one constant region. It is known in the art that the variable region of the heavy and light chain region of an antibody is for antigen binding. In one example of the use disclosed herein, the variable region comprises three complementarity-determining regions (i.e., CDR1, CDR2 and CDR3) and four framework regions (i.e., FR1, FR2, FR3 and FR4). In one example of the use disclosed herein, the complementarity-determining regions and framework regions are in the order of FR1-CDR1-FR2-CDR2-FR3-CDR3-FR4.

In one example, the antibody or antigen-binding fragment thereof for treating TBI or for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the antibody or antigen-binding fragment thereof for treating TBI or for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12. In one example, the antibody or antigen-binding fragment thereof for treating TBI or for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13. In one example, the antibody or antigen-binding fragment thereof for treating TBI or for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14. In one example, the antibody or antigen-binding fragment thereof for treating TBT or for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the antibody or antigen-binding fragment thereof for treating TBI or for use in the manufacture of a medicament for treating TBI comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

In one example, the antibody or antigen-binding fragment thereof for treating TBI or for use in the manufacture of a medicament for treating TBI, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A2. In one example, the antibody or antigen-binding fragment thereof for treating TBI or for use in the manufacture of a medicament for treating TBI, having a heavy chain variable region comprising an amino acid sequence of SEQ ID NO: 7, and a light chain variable region comprising an amino acid sequence of SEQ ID NO: 11 corresponds to humanized antibody clone A2. In one example, the antibody or antigen-binding fragment thereof for treating TBI or for use in the manufacture of a medicament for treating TBI, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A3. In one example, the antibody or antigen-binding fragment thereof for treating TBI or for use in the manufacture of a medicament for treating TBI, having a heavy chain variable region comprising an amino acid sequence of SEQ ID NO: 7, and a light chain variable region comprising an amino acid sequence of SEQ ID NO: 12 corresponds to humanized antibody clone A3. In one example, the antibody or antigen-binding fragment thereof for treating TBI or for use in the manufacture of a medicament for treating TBI, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13 corresponds to humanized antibody clone A4. In one example, the antibody or antigen-binding fragment thereof for treating TBI or for use in the manufacture of a medicament for treating TBI, having a heavy chain variable region comprising an amino acid sequence of SEQ ID NO: 7, and a light chain variable region comprising an amino acid sequence of SEQ ID NO: 13 corresponds to humanized antibody clone A4. In one example, the antibody or antigen-binding fragment thereof for treating TBI or for use in the manufacture of a medicament for treating TBI, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14 corresponds to humanized antibody clone A5. In one example, the antibody or antigen-binding fragment thereof for treating TBI or for use in the manufacture of a medicament for treating TBI, having a heavy chain variable region comprising an amino acid sequence of SEQ ID NO: 7, and a light chain variable region comprising an amino acid sequence of SEQ ID NO: 14 corresponds to humanized antibody clone A5. In one example, the antibody or antigen-binding fragment thereof for treating TBI or for use in the manufacture of a medicament for treating TBI, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6. In one example, the antibody or antigen-binding fragment thereof for treating TBI or for use in the manufacture of a medicament for treating TBI, having a heavy chain variable region comprising an amino acid sequence of SEQ ID NO: 8, and a light chain variable region comprising an amino acid sequence of SEQ ID NO: 11 corresponds to humanized antibody clone A6. In one example, the antibody or antigen-binding fragment thereof for treating TBI or for use in the manufacture of a medicament for treating TBI, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A7. In one example, the antibody or antigen-binding fragment thereof for treating TBI or for use in the manufacture of a medicament for treating TBI, having a heavy chain variable region comprising an amino acid sequence of SEQ ID NO: 8, and a light chain variable region comprising an amino acid sequence of SEQ ID NO: 12 corresponds to humanized antibody clone A7.

In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO. 55, or SEQ ID NO: 56.

In one example, the antibody or antigen-binding fragment thereof for treating TBI or for use in the manufacture of a medicament for treating TBI comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59. In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60. In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating TBI or for use in the manufacture of a medicament for treating TBI comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58.

In one example, the antibody or antigen-binding fragment thereof for treating TBI or for use in the manufacture of a medicament for treating TBI, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A2. In one example, the antibody or antigen-binding fragment thereof for treating TBI or for use in the manufacture of a medicament for treating TBI, having a heavy chain comprising an amino acid sequence of SEQ ID NO: 50, and a light chain comprising an amino acid sequence of SEQ ID NO: 57 corresponds to humanized antibody clone A2. In one example, the antibody or antigen-binding fragment thereof for treating TBI or for use in the manufacture of a medicament for treating TBL, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A3. In one example, the antibody or antigen-binding fragment thereof for treating TBI or for use in the manufacture of a medicament for treating TBI, having a heavy chain comprising an amino acid sequence of SEQ ID NO: 50, and a light chain comprising an amino acid sequence of SEQ ID NO: 58 corresponds to humanized antibody clone A3. In one example, the antibody or antigen-binding fragment thereof for treating TBI or for use in the manufacture of a medicament for treating TBI, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59 corresponds to humanized antibody clone A4. In one example, the antibody or antigen-binding fragment thereof for treating TBI or for use in the manufacture of a medicament for treating TBI, having a heavy chain comprising an amino acid sequence of SEQ ID NO: 50, and a light chain comprising an amino acid sequence of SEQ ID NO: 59 corresponds to humanized antibody clone A4. In one example, the antibody or antigen-binding fragment thereof for treating TBI or for use in the manufacture of a medicament for treating TBI, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60 corresponds to humanized antibody clone A5. In one example, the antibody or antigen-binding fragment thereof for treating TBI or for use in the manufacture of a medicament for treating TBI, having a heavy chain comprising an amino acid sequence of SEQ ID NO: 50, and a light chain comprising an amino acid sequence of SEQ ID NO: 60 corresponds to humanized antibody clone A5. In one example, the antibody or antigen-binding fragment thereof for treating TBI or for use in the manufacture of a medicament for treating TBI, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6. In one example, the antibody or antigen-binding fragment thereof for treating TBI or for use in the manufacture of a medicament for treating TBI, having a heavy chain comprising an amino acid sequence of SEQ ID NO: 51, and a light chain comprising an amino acid sequence of SEQ ID NO: 57 corresponds to humanized antibody clone A6. In one example, the antibody or antigen-binding fragment thereof for treating TBI or for use in the manufacture of a medicament for treating TBI, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A7. In one example, the antibody or antigen-binding fragment thereof for treating TBI or for use in the manufacture of a medicament for treating TBI, having a heavy chain comprising an amino acid sequence of SEQ ID NO: 51, and a light chain comprising an amino acid sequence of SEQ ID NO: 58 corresponds to humanized antibody clone A7.

In one example, the antibody or antigen-binding fragment thereof for treating TBI or for use in the manufacture of a medicament for treating TBI comprises a constant region selected from the major classes of immunoglobulins, such as IgG, IgD, IgE, IgA and IgM. In one example, the constant region is selected from the group consisting of IgG1, IgG2, IgG3, and IgG4. In one example, the constant region is IgG1. In another example, the constant region is IgG4. In one example, the antibody or antigen-binding fragment thereof for treating TBI or for use in the manufacture of a medicament for treating TBI comprises a heavy chain comprising an IgG1 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO; 50, SEQ ID NO: 51, SEQ ID NO: 52, SEQ ID NO: 53, SEQ ID NO: 54 or SEQ ID NO: 55. In one example, the antibody or antigen-binding fragment thereof for treating TBI or for use in the manufacture of a medicament for treating TBI comprises a heavy chain comprising an IgG4 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity SEQ ID NO: 56.

In one example, the antibody or antigen-binding fragment thereof for treating TBI or for use in the manufacture of a medicament for treating TBI specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

In one example, the antibody or antigen-binding fragment thereof for treating TBI or for use in the manufacture of a medicament for treating TBI is encoded by a nucleic acid comprising a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63. In one example, the antibody or antigen-binding fragment thereof for treating TBI or for use in the manufacture of a medicament for treating TBI, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively. In one example, the antibody or antigen-binding fragment thereof for treating TBI or for use in the manufacture of a medicament for treating TBI, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively.

In one example, the antibody or antigen-binding fragment thereof for treating TBI or for use in the manufacture of a medicament for treating TBI, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively. In one example, the antibody or antigen-binding fragment thereof for treating TBI or for use in the manufacture of a medicament for treating TBI, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively. In one example, the antibody or antigen-binding fragment thereof for treating TBI or for use in the manufacture of a medicament for treating TBI, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively. In one example, the antibody or antigen-binding fragment thereof for treating TBI or for use in the manufacture of a medicament for treating TBI, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively. In one example, the antibody or antigen-binding fragment thereof for treating TBI or for use in the manufacture of a medicament for treating TBI, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 61. In one example, the antibody or antigen-binding fragment thereof for treating TBI or for use in the manufacture of a medicament for treating TBI, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 62. In one example, the antibody or antigen-binding fragment thereof for treating TBI or for use in the manufacture of a medicament for treating TBI, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 63.

In one example, the antibody or antigen-binding fragment thereof for treating TBI or for use in the manufacture of a medicament for treating TBI, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively corresponds to humanized antibody clone A2. In one example, the antibody or antigen-binding fragment thereof for treating TBI or for use in the manufacture of a medicament for treating TBI, having its heavy chain and its light chain encoded by polynucleotide sequences of SEQ ID NOs: 17 and 21, respectively, corresponds to humanized antibody clone A2. In one example, the antibody or antigen-binding fragment thereof for treating TBI or for use in the manufacture of a medicament for treating TBI, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively corresponds to humanized antibody clone A3. In one example, the antibody or antigen-binding fragment thereof for treating TBI or for use in the manufacture of a medicament for treating TBI, having its heavy chain and its light chain encoded by polynucleotide sequences of SEQ ID NOs: 17 and 22, respectively, corresponds to humanized antibody clone A3. In one example, the antibody or antigen-binding fragment thereof for treating TBI or for use in the manufacture of a medicament for treating TBI, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively corresponds to humanized antibody clone A4. In one example, the antibody or antigen-binding fragment thereof for treating TBI or for use in the manufacture of a medicament for treating TBI, having its heavy chain and its light chain encoded by polynucleotide sequences of SEQ ID NOs: 17 and 23, respectively, corresponds to humanized antibody clone A4. In one example, the antibody or antigen-binding fragment thereof for treating TBI or for use in the manufacture of a medicament for treating TBI, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively corresponds to humanized antibody clone A5. In one example, the antibody or antigen-binding fragment thereof for treating TBI or for use in the manufacture of a medicament for treating TBI, having its heavy chain and its light chain encoded by polynucleotide sequences of SEQ ID NOs: 17 and 24, respectively, corresponds to humanized antibody clone A5. In one example, the antibody or antigen-binding fragment thereof for treating TBI or for use in the manufacture of a medicament for treating TBI, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively corresponds to humanized antibody clone A6. In one example, the antibody or antigen-binding fragment thereof for treating TBI or for use in the manufacture of a medicament for treating TBI, having its heavy chain and its light chain encoded by polynucleotide sequences of SEQ ID NOs: 18 and 21, respectively, corresponds to humanized antibody clone A6. In one example, the antibody or antigen-binding fragment thereof for treating TBI or for use in the manufacture of a medicament for treating TBI, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively corresponds to humanized antibody clone A7. In one example, the antibody or antigen-binding fragment thereof for treating TBI or for use in the manufacture of a medicament for treating TBI, having its heavy chain and its light chain encoded by polynucleotide sequences of SEQ ID NOs: 18 and 22, respectively, corresponds to humanized antibody clone A7.

In one example, the effective amount of the antibody or antigen-binding fragment thereof for treating TBI or for use in the manufacture of a medicament for treating TBI is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg. Generally, an effective dosage may be in the range of about 0.1 mg to about 15 mg per kg body weight of the subject; about 0.2 mg to about 14 mg per kg body weight of the subject; about 0.4 mg to about 13 mg per kg body weight of the subject; about 0.6 mg to about 12 mg per kg body weight of the subject; about 0.8 mg to about 11 mg per kg body weight of the subject; or about 1 mg to about 10 mg per kg body weight of the subject; or about 2 mg to about 9 mg per kg body weight of the subject; about 3 mg to about 8 mg per kg body weight of the subject; about 4 mg to about 7 mg per kg body weight of the subject; or about 0.1 mg per kg body weight of the subject, or about 0.2 mg per kg body weight of the subject; or about 0.4 mg per kg body weight of the subject, or about 0.6 mg per kg body weight of the subject, or about 0.8 mg per kg body weight of the subject, or about 1 mg per kg body weight of the subject, or about 2 mg per kg body weight of the subject, or about 3 mg per kg body weight of the subject, or about 4 mg per kg body weight of the subject, or about 5 mg per kg body weight of the subject, or about 6 mg per kg body weight of the subject, or about 7 mg per kg body weight of the subject, or about 8 mg per kg body weight of the subject, or about 9 mg per kg body weight of the subject, or about 10 mg per kg body weight of the subject, or about 11 mg per kg body weight of the subject, or about 12 mg per kg body weight of the subject, or about 13 mg per kg body weight of the subject, or about 14 mg per kg body weight of the subject, or about 15 mg per kg body weight of the subject.

In one example, the TBI is a result of one or more physical injury events. In one example, the physical injury event is selected from the group consisting of collision, vehicle-related incident, fall, sport-related incident, military-related incident and explosion-related incident. In one example, the military-related incident is a military attack. In one example, the explosion-related incident is bomb explosion. In one example, the physical injury event is vehicle-related incident. In one example, the physical injury event is fall. In one example, the physical injury events are vehicle-related incident and fall.

In one example, the antibody or antigen-binding fragment thereof for treating TBI or for use in the manufacture of a medicament for treating TBI inhibits TRPM4 activity. In one example, the antibody or antigen-binding fragment thereof for treating TBI or for use in the manufacture of a medicament for treating TBI inhibits TRPM4 activity by inhibiting TRPM4 current. In one example, the antibody or antigen-binding fragment thereof for treating TBI or for use in the manufacture of a medicament for treating TBI inhibits TRPM4 activity by internalizing membrane TRPM4 protein. In one example, the antibody or antigen-binding fragment thereof for treating TBI or for use in the manufacture of a medicament for treating TBI inhibits TRPM4 activity by inhibiting TRPM4 current and internalizing membrane TRPM4 protein. In one example, under diseased conditions, inhibiting TRPM4 activity refers to blocking, preventing or disrupting the upregulated or activated protein function of TRPM4.

In one example, the effective amount of the antibody or antigen-binding fragment thereof treats TBI by preventing damage to the blood-brain barrier and/or a neuron, and/or by alleviating the damage to the blood-brain barrier and/or a neuron. In one example, the effective amount of the antibody or antigen-binding fragment thereof treats TBI by preventing damage to the blood-brain barrier and/or a neuron. In one example, the effective amount of the antibody or antigen-binding fragment thereof treats TBI by preventing damage to the blood-brain barrier. In one example, the effective amount of the antibody or antigen-binding fragment thereof treats TBI by preventing damage to a neuron. In one example, the effective amount of the antibody or antigen-binding fragment thereof treats TBI by preventing damage to the blood-brain barrier and a neuron. In one example, the effective amount of the antibody or antigen-binding fragment thereof treats TBI by alleviating the damage to the blood-brain barrier and/or a neuron. In one example, the effective amount of the antibody or antigen-binding fragment thereof treats TBI by alleviating the damage to the blood-brain barrier. In one example, the effective amount of the antibody or antigen-binding fragment thereof treats TBI by alleviating the damage to a neuron. In one example, the effective amount of the antibody or antigen-binding fragment thereof treats TBI by alleviating the damage to the blood-brain barrier and a neuron. As used herein, the term “preventing”, and grammatical variants thereof, means to hinder or stop a problem or situation or condition or phenomenon or an event from occurring or happening or developing. For example, the term “preventing”, and grammatical variants thereof, in the context of TBI treatment means to hinder or stop the damage to the blood brain barrier and/or a neuron from occurring or happening or developing. As used herein, the term “alleviating”, and grammatical variants thereof, means to delay, lessen or reduce the progression or continued development or severity or worsening of a problem or situation or condition or phenomenon or an event. For example, the term “alleviating”, and grammatical variants thereof, in the context of TBI treatment means to delay, lessen, or reduce the progression or continued development or severity or worsening of the damage to the blood brain barrier and/or a neuron. In one example, if damage to the blood-brain barrier is occurring or happening or developing, the humanized monoclonal antibody or antigen-binding fragment thereof disclosed herein has an effect or is able to stop or delay the damaging process.

In one example, the effective amount of the antibody or antigen-binding fragment thereof treats TBI by reducing one or more symptoms of TBI in the subject. In one example, the symptom of TBI is selected from the group consisting of confusion, dizziness, sleep disturbances, breathing difficulties, anxiety, loss of consciousness, severe headache, repeated nausea and vomiting, slurred speech, walking difficulties, weakness in one side or part of the body, loss of sensory to one side or part of the body, vegetative state, and locked-in syndrome. In one example, the symptom of TBI is associated with hypoxia.

In one example, the method of treating TBI as disclosed herein further comprises administering to the subject one or more interventions selected from the group consisting of: (a) one or more surgical inventions; and (b) one or more therapeutic agents. In one example, the method of treating TBI as disclosed herein further comprises administering to the subject one or more surgical interventions. In one example, the method of treating TBI as disclosed herein further comprises administering to the subject one or more therapeutic agents. In one example, the method of treating TBI as disclosed herein further comprises administering to the subject one or more surgical interventions and one or more therapeutic agents. In one example of the use disclosed herein, the subject is to be further administered one or more interventions selected from the group consisting of: (a) one or more surgical inventions; and (b) one or more therapeutic agents. In one example of the use disclosed herein, the subject is to be further administered one or more surgical interventions. In one example of the use disclosed herein, the subject is to be further administered one or more therapeutic agents. In one example of the use disclosed herein, the subject is to be further administered one or more surgical interventions and one or more therapeutic agents. The surgical intervention may include any surgical interventions known in the art for treating TBI, such as, but is not limited to, the removal of hematoma, mass lesion and/or cerebrospinal fluid, and bony decompression. In one example, the surgical intervention may include any surgical interventions that treats TBI by preventing damage to the blood-brain barrier, preventing damage to a neuron, alleviating damage to the blood-brain barrier, alleviating damage to a neuron and/or reducing one or more symptoms of TBI in the subject. The therapeutic agent for further administration in the treatment of TBI may include any agents, such as small molecule drug, antibody, oligonucleotide drug, etc. In one example, the therapeutic agent for further administration may include any agents that treats TBI by preventing damage to the blood-brain barrier, preventing damage to a neuron, alleviating damage to the blood-brain barrier, alleviating damage to a neuron and/or reducing one or more symptoms of TBI in the subject. In one example, the therapeutic agent for further administration reduces the edema or swelling or inflammation in TBI. In one example, the therapeutic agent for further administration may include, but is not limited to, non-steroidal anti-inflammatory drugs, salicylates, corticosteroids, and mannitol.

In one example, the method of treating TBI as disclosed herein further comprises (a) monitoring the subject by using a multimodality monitoring program; and/or (b) providing neurocritical care to the subject. In one example, the method of treating TBI as disclosed herein further comprises monitoring the subject by using a multimodality monitoring program. In one example, the method of treating TBI as disclosed herein further comprises providing neurocritical care to the subject. In one example, the method of treating TBI as disclosed herein further comprises monitoring the subject by using a multimodality monitoring program and providing neurocritical care to the subject. In one example of the use disclosed herein, (a) the subject is to be further monitored by using a multimodality monitoring program; and/or (b) neurocritical care is to be further provided to the subject. In one example of the use disclosed herein, the subject is to be further monitored by using a multimodality monitoring program. In one example of the use disclosed herein, neurocritical care is to be further provided to the subject. In one example of the use disclosed herein, the subject is to be further monitored by using a multimodality monitoring program and neurocritical care is to be further provided to the subject. In one example, the neurocritical care refers to intensive care. In one example, the neurocritical care refers to intensive care which is specialized for patient with neurological disease. The multimodality monitoring program or neurocritical care or intensive care allows for the detection of one or more symptoms that may develop before, during, or after treatment with the antibody or antigen-binding fragment thereof disclosed herein, or the pharmaceutical composition disclosed herein. In one example, the method of treating TBI as disclosed herein further comprises administering to the subject an effective amount of the antibody or antigen-binding fragment thereof disclosed herein if one or more symptoms are detected. In one example of the use disclosed herein, the subject is to be further administered an effective amount of the antibody or antigen-binding fragment thereof disclosed herein if one or more symptoms are detected.

In one example, the present disclosure refers to a kit for treating TBI comprising the antibody or antigen-binding fragment thereof disclosed herein and/or instructions for use. In some examples, the reagents provided in the kit as described herein may be provided in separate containers comprising the components independently distributed in one or more containers.

In one example, the present disclosure refers to a method of treating vascular dementia, comprising administering to a subject an effective amount of the antibody or antigen-binding fragment thereof disclosed herein, or the pharmaceutical composition disclosed herein. In one example, the present disclosure refers to a method of treating vascular dementia, comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6. In one example, the antibody or antigen-binding fragment thereof for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example of the method disclosed herein, each heavy and light chain of the antibody or antigen-binding fragment thereof comprises one variable region and one constant region. It is known in the art that the variable region of the heavy and light chain region of an antibody is for antigen binding. In one example of the method disclosed herein, the variable region comprises three complementarity-determining regions (i.e., CDR1, CDR2 and CDR3) and four framework regions (i.e., FR1, FR2, FR3 and FR4). In one example of the method disclosed herein, the complementarity-determining regions and framework regions are in the order of FR1-CDR1-FR2-CDR2-FR3-CDR3-FR4. The term “vascular dementia” refers to a type of dementia typically caused by a reduction in the blood flow to the brain.

In one example, the present disclosure refers to use of an effective amount of the antibody or antigen-binding fragment thereof disclosed herein, or the pharmaceutical composition disclosed herein, in the manufacture of a medicament for treating vascular dementia. In one example, the present disclosure refers to use of an effective amount of a humanized monoclonal antibody or antigen-binding fragment thereof specific to a transient receptor potential melastatin 4 (TRPM4) protein in the manufacture of a medicament for treating vascular dementia, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6. In one example, the antibody or antigen-binding fragment thereof for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example of the use disclosed herein, each heavy and light chain of the antibody or antigen-binding fragment thereof comprises one variable region and one constant region. It is known in the art that the variable region of the heavy and light chain region of an antibody is for antigen binding. In one example of the use disclosed herein, the variable region comprises three complementarity-determining regions (i.e., CDR1, CDR2 and CDR3) and four framework regions (i.e., FR1, FR2, FR3 and FR4). In one example of the use disclosed herein, the complementarity-determining regions and framework regions are in the order of FR1-CDR1-FR2-CDR2-FR3-CDR3-FR4.

In one example, the antibody or antigen-binding fragment thereof for treating vascular dementia or for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the antibody or antigen-binding fragment thereof for treating vascular dementia or for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12. In one example, the antibody or antigen-binding fragment thereof for treating vascular dementia or for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13. In one example, the antibody or antigen-binding fragment thereof for treating vascular dementia or for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14. In one example, the antibody or antigen-binding fragment thereof for treating vascular dementia or for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the antibody or antigen-binding fragment thereof for treating vascular dementia or for use in the manufacture of a medicament for treating vascular dementia comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

In one example, the antibody or antigen-binding fragment thereof for treating vascular dementia or for use in the manufacture of a medicament for treating vascular dementia, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A2. In one example, the antibody or antigen-binding fragment thereof for treating vascular dementia or for use in the manufacture of a medicament for treating vascular dementia, having a heavy chain variable region comprising an amino acid sequence of SEQ ID NO: 7, and a light chain variable region comprising an amino acid sequence of SEQ ID NO: 11 corresponds to humanized antibody clone A2. In one example, the antibody or antigen-binding fragment thereof for treating vascular dementia or for use in the manufacture of a medicament for treating vascular dementia, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A3. In one example, the antibody or antigen-binding fragment thereof for treating vascular dementia or for use in the manufacture of a medicament for treating vascular dementia, having a heavy chain variable region comprising an amino acid sequence of SEQ ID NO: 7, and a light chain variable region comprising an amino acid sequence of SEQ ID NO: 12 corresponds to humanized antibody clone A3. In one example, the antibody or antigen-binding fragment thereof for treating vascular dementia or for use in the manufacture of a medicament for treating vascular dementia, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13 corresponds to humanized antibody clone A4. In one example, the antibody or antigen-binding fragment thereof for treating vascular dementia or for use in the manufacture of a medicament for treating vascular dementia, having a heavy chain variable region comprising an amino acid sequence of SEQ ID NO: 7, and a light chain variable region comprising an amino acid sequence of SEQ ID NO: 13 corresponds to humanized antibody clone A4. In one example, the antibody or antigen-binding fragment thereof for treating vascular dementia or for use in the manufacture of a medicament for treating vascular dementia, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14 corresponds to humanized antibody clone A5. In one example, the antibody or antigen-binding fragment thereof for treating vascular dementia or for use in the manufacture of a medicament for treating vascular dementia, having a heavy chain variable region comprising an amino acid sequence of SEQ ID NO: 7, and a light chain variable region comprising an amino acid sequence of SEQ ID NO: 14 corresponds to humanized antibody clone A5. In one example, the antibody or antigen-binding fragment thereof for treating vascular dementia or for use in the manufacture of a medicament for treating vascular dementia, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6. In one example, the antibody or antigen-binding fragment thereof for treating vascular dementia or for use in the manufacture of a medicament for treating vascular dementia, having a heavy chain variable region comprising an amino acid sequence of SEQ ID NO: 8, and a light chain variable region comprising an amino acid sequence of SEQ ID NO: 11 corresponds to humanized antibody clone A6. In one example, the antibody or antigen-binding fragment thereof for treating vascular dementia or for use in the manufacture of a medicament for treating vascular dementia, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A7. In one example, the antibody or antigen-binding fragment thereof for treating vascular dementia or for use in the manufacture of a medicament for treating vascular dementia, having a heavy chain variable region comprising an amino acid sequence of SEQ ID NO: 8, and a light chain variable region comprising an amino acid sequence of SEQ ID NO: 12 corresponds to humanized antibody clone A7.

In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO. 55, or SEQ ID NO: 56.

In one example, the antibody or antigen-binding fragment thereof for treating vascular dementia or for use in the manufacture of a medicament for treating vascular dementia comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59. In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60. In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating vascular dementia or for use in the manufacture of a medicament for treating vascular dementia comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58.

In one example, the antibody or antigen-binding fragment thereof for treating vascular dementia or for use in the manufacture of a medicament for treating vascular dementia, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A2. In one example, the antibody or antigen-binding fragment thereof for treating vascular dementia or for use in the manufacture of a medicament for treating vascular dementia, having a heavy chain comprising an amino acid sequence of SEQ ID NO: 50, and a light chain comprising an amino acid sequence of SEQ ID NO: 57 corresponds to humanized antibody clone A2. In one example, the antibody or antigen-binding fragment thereof for treating vascular dementia or for use in the manufacture of a medicament for treating vascular dementia, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A3. In one example, the antibody or antigen-binding fragment thereof for treating vascular dementia or for use in the manufacture of a medicament for treating vascular dementia, having a heavy chain comprising an amino acid sequence of SEQ ID NO: 50, and a light chain comprising an amino acid sequence of SEQ ID NO: 58 corresponds to humanized antibody clone A3. In one example, the antibody or antigen-binding fragment thereof for treating vascular dementia or for use in the manufacture of a medicament for treating vascular dementia, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59 corresponds to humanized antibody clone A4. In one example, the antibody or antigen-binding fragment thereof for treating vascular dementia or for use in the manufacture of a medicament for treating vascular dementia, having a heavy chain comprising an amino acid sequence of SEQ ID NO: 50, and a light chain comprising an amino acid sequence of SEQ ID NO: 59 corresponds to humanized antibody clone A4. In one example, the antibody or antigen-binding fragment thereof for treating vascular dementia or for use in the manufacture of a medicament for treating vascular dementia, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60 corresponds to humanized antibody clone A5. In one example, the antibody or antigen-binding fragment thereof for treating vascular dementia or for use in the manufacture of a medicament for treating vascular dementia, having a heavy chain comprising an amino acid sequence of SEQ ID NO: 50, and a light chain comprising an amino acid sequence of SEQ ID NO: 60 corresponds to humanized antibody clone A5. In one example, the antibody or antigen-binding fragment thereof for treating vascular dementia or for use in the manufacture of a medicament for treating vascular dementia, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6. In one example, the antibody or antigen-binding fragment thereof for treating vascular dementia or for use in the manufacture of a medicament for treating vascular dementia, having a heavy chain comprising an amino acid sequence of SEQ ID NO: 51, and a light chain comprising an amino acid sequence of SEQ ID NO: 57 corresponds to humanized antibody clone A6. In one example, the antibody or antigen-binding fragment thereof for treating vascular dementia or for use in the manufacture of a medicament for treating vascular dementia, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A7. In one example, the antibody or antigen-binding fragment thereof for treating vascular dementia or for use in the manufacture of a medicament for treating vascular dementia, having a heavy chain comprising an amino acid sequence of SEQ ID NO: 51, and a light chain comprising an amino acid sequence of SEQ ID NO: 58 corresponds to humanized antibody clone A7.

In one example, the antibody or antigen-binding fragment thereof for treating vascular dementia or for use in the manufacture of a medicament for treating vascular dementia comprises a constant region selected from the major classes of immunoglobulins, such as IgG, IgD, IgE, IgA and IgM. In one example, the constant region is selected from the group consisting of IgG1, IgG2, IgG3, and IgG4. In one example, the constant region is IgG1. In another example, the constant region is IgG4. In one example, the antibody or antigen-binding fragment thereof for treating vascular dementia or for use in the manufacture of a medicament for treating vascular dementia comprises a heavy chain comprising an IgG1 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54 or SEQ ID NO: 55. In one example, the antibody or antigen-binding fragment thereof for treating vascular dementia or for use in the manufacture of a medicament for treating vascular dementia comprises a heavy chain comprising an IgG4 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity SEQ ID NO: 56.

In one example, the antibody or antigen-binding fragment thereof for treating vascular dementia or for use in the manufacture of a medicament for treating vascular dementia specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

In one example, the antibody or antigen-binding fragment thereof for treating vascular dementia or for use in the manufacture of a medicament for treating vascular dementia is encoded by a nucleic acid comprising a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63. In one example, the antibody or antigen-binding fragment thereof for treating vascular dementia or for use in the manufacture of a medicament for treating vascular dementia, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively. In one example, the antibody or antigen-binding fragment thereof for treating vascular dementia or for use in the manufacture of a medicament for treating vascular dementia, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively. In one example, the antibody or antigen-binding fragment thereof for treating vascular dementia or for use in the manufacture of a medicament for treating vascular dementia, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively. In one example, the antibody or antigen-binding fragment thereof for treating vascular dementia or for use in the manufacture of a medicament for treating vascular dementia, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively. In one example, the antibody or antigen-binding fragment thereof for treating vascular dementia or for use in the manufacture of a medicament for treating vascular dementia, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively. In one example, the antibody or antigen-binding fragment thereof for treating vascular dementia or for use in the manufacture of a medicament for treating vascular dementia, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively. In one example, the antibody or antigen-binding fragment thereof for treating vascular dementia or for use in the manufacture of a medicament for treating vascular dementia, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 61. In one example, the antibody or antigen-binding fragment thereof for treating vascular dementia or for use in the manufacture of a medicament for treating vascular dementia, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 62. In one example, the antibody or antigen-binding fragment thereof for treating vascular dementia or for use in the manufacture of a medicament for treating vascular dementia, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 63.

In one example, the antibody or antigen-binding fragment thereof for treating vascular dementia or for use in the manufacture of a medicament for treating vascular dementia, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively corresponds to humanized antibody clone A2. In one example, the antibody or antigen-binding fragment thereof for treating vascular dementia or for use in the manufacture of a medicament for treating vascular dementia, having its heavy chain and its light chain encoded by polynucleotide sequences of SEQ ID NOs: 17 and 21, respectively, corresponds to humanized antibody clone A2. In one example, the antibody or antigen-binding fragment thereof for treating vascular dementia or for use in the manufacture of a medicament for treating vascular dementia, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively corresponds to humanized antibody clone A3. In one example, the antibody or antigen-binding fragment thereof for treating vascular dementia or for use in the manufacture of a medicament for treating vascular dementia, having its heavy chain and its light chain encoded by polynucleotide sequences of SEQ ID NOs: 17 and 22, respectively, corresponds to humanized antibody clone A3. In one example, the antibody or antigen-binding fragment thereof for treating vascular dementia or for use in the manufacture of a medicament for treating vascular dementia, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively corresponds to humanized antibody clone A4. In one example, the antibody or antigen-binding fragment thereof for treating vascular dementia or for use in the manufacture of a medicament for treating vascular dementia, having its heavy chain and its light chain encoded by polynucleotide sequences of SEQ ID NOs: 17 and 23, respectively, corresponds to humanized antibody clone A4. In one example, the antibody or antigen-binding fragment thereof for treating vascular dementia or for use in the manufacture of a medicament for treating vascular dementia, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively corresponds to humanized antibody clone A5. In one example, the antibody or antigen-binding fragment thereof for treating vascular dementia or for use in the manufacture of a medicament for treating vascular dementia, having its heavy chain and its light chain encoded by polynucleotide sequences of SEQ ID NOs: 17 and 24, respectively, corresponds to humanized antibody clone A5. In one example, the antibody or antigen-binding fragment thereof for treating vascular dementia or for use in the manufacture of a medicament for treating vascular dementia, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively corresponds to humanized antibody clone A6. In one example, the antibody or antigen-binding fragment thereof for treating vascular dementia or for use in the manufacture of a medicament for treating vascular dementia, having its heavy chain and its light chain encoded by polynucleotide sequences of SEQ ID NOs: 18 and 21, respectively, corresponds to humanized antibody clone A6. In one example, the antibody or antigen-binding fragment thereof for treating vascular dementia or for use in the manufacture of a medicament for treating vascular dementia, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively corresponds to humanized antibody clone A7. In one example, the antibody or antigen-binding fragment thereof for treating vascular dementia or for use in the manufacture of a medicament for treating vascular dementia, having its heavy chain and its light chain encoded by polynucleotide sequences of SEQ ID NOs: 18 and 22, respectively, corresponds to humanized antibody clone A7.

In one example, the effective amount of the antibody or antigen-binding fragment thereof for treating vascular dementia or for use in the manufacture of a medicament for treating vascular dementia is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg. Generally, an effective dosage may be in the range of about 0.1 mg to about 15 mg per kg body weight of the subject; about 0.2 mg to about 14 mg per kg body weight of the subject; about 0.4 mg to about 13 mg per kg body weight of the subject; about 0.6 mg to about 12 mg per kg body weight of the subject; about 0.8 mg to about 11 mg per kg body weight of the subject; or about 1 mg to about 10 mg per kg body weight of the subject; or about 2 mg to about 9 mg per kg body weight of the subject; about 3 mg to about 8 mg per kg body weight of the subject; about 4 mg to about 7 mg per kg body weight of the subject; or about 0.1 mg per kg body weight of the subject, or about 0.2 mg per kg body weight of the subject; or about 0.4 mg per kg body weight of the subject, or about 0.6 mg per kg body weight of the subject, or about 0.8 mg per kg body weight of the subject, or about 1 mg per kg body weight of the subject, or about 2 mg per kg body weight of the subject, or about 3 mg per kg body weight of the subject, or about 4 mg per kg body weight of the subject, or about 5 mg per kg body weight of the subject, or about 6 mg per kg body weight of the subject, or about 7 mg per kg body weight of the subject, or about 8 mg per kg body weight of the subject, or about 9 mg per kg body weight of the subject, or about 10 mg per kg body weight of the subject, or about 11 mg per kg body weight of the subject, or about 12 mg per kg body weight of the subject, or about 13 mg per kg body weight of the subject, or about 14 mg per kg body weight of the subject, or about 15 mg per kg body weight of the subject.

In one example, the antibody or antigen-binding fragment thereof for treating vascular dementia or for use in the manufacture of a medicament for treating vascular dementia inhibits TRPM4 activity. In one example, the antibody or antigen-binding fragment thereof for treating vascular dementia or for use in the manufacture of a medicament for treating vascular dementia inhibits TRPM4 activity by inhibiting TRPM4 current. In one example, the antibody or antigen-binding fragment thereof for treating vascular dementia or for use in the manufacture of a medicament for treating vascular dementia inhibits TRPM4 activity by internalizing membrane TRPM4 protein. In one example, the antibody or antigen-binding fragment thereof for treating vascular dementia or for use in the manufacture of a medicament for treating vascular dementia inhibits TRPM4 activity by inhibiting TRPM4 current and internalizing membrane TRPM4 protein. In one example, under diseased conditions, inhibiting TRPM4 activity refers to blocking, preventing or disrupting the upregulated or activated protein function of TRPM4.

In one example, the effective amount of antibody or antigen-binding fragment thereof treats vascular dementia by improving the cognitive function in the subject. In one example, the cognitive function is selected from the group consisting of memory, learning, thinking and perception. As used herein, the term “improving”, and grammatical variants thereof, means enhancing or increasing or making a situation or condition or ability or function to be in a better state compared to a control or reference situation or condition or ability or function. For example, the term “improving”, and grammatical variants thereof, in the context of treatment of vascular dementia means enhancing or increasing or making the subject's ability or function (such as cognitive function) to be in a better state compared to that of an untreated subject.

In one example, the effective amount of the antibody or antigen-binding fragment thereof treats vascular dementia by reducing one or more symptoms of vascular dementia in the subject. In one example, the symptom of vascular dementia is selected from the group consisting of confusion, dizziness, sleep disturbances, breathing difficulties, anxiety, difficulty in paying attention, difficulty in concentrating, difficulty in organization, reduced ability to organize thoughts or actions, memory impairment, restlessness, agitation, depression and apathy. In one example, the symptom of vascular dementia is associated with hypoxia.

In one example, the vascular dementia is a result of one or more disorders selected from the group consisting of atherosclerosis, cerebral amyloid angiopathy (CAA), genetic small vessel angiopathy, inflammation, immune-mediated small vessel disease, venous collagenosis, and obesity.

In one example, the method of treating vascular dementia as disclosed herein further comprises administering to the subject one or more interventions selected from the group consisting of: (a) one or more surgical inventions; and (b) one or more therapeutic agents. In one example, the method of treating vascular dementia as disclosed herein further comprises administering to the subject one or more surgical interventions. In one example, the method of treating vascular dementia as disclosed herein further comprises administering to the subject one or more therapeutic agents. In one example, the method of treating vascular dementia as disclosed herein further comprises administering to the subject one or more surgical interventions and one or more therapeutic agents. In one example of the use disclosed herein, the subject is to be further administered one or more interventions selected from the group consisting of: (a) one or more surgical inventions; and (b) one or more therapeutic agents. In one example of the use disclosed herein, the subject is to be further administered one or more surgical interventions. In one example of the use disclosed herein, the subject is to be further administered one or more therapeutic agents. In one example of the use disclosed herein, the subject is to be further administered one or more surgical interventions and one or more therapeutic agents. The surgical intervention may include any surgical interventions known in the art for treating vascular dementia, such as, but is not limited to, carotid endarterectomy (CEA) and carotid angioplasty and stenting (CAS). In one example, the surgical intervention is carotid endarterectomy (CEA). In one example, the surgical intervention is carotid angioplasty and stenting (CAS). In one example, the surgical intervention may include any surgical interventions that treats vascular dementia by improving the cognitive function and/or reducing one or more symptoms of vascular dementia in the subject. The therapeutic agent for further administration may include any agents to manage or treat a subject with vascular dementia, such as small molecule drug, antibody, oligonucleotide drug, etc. In one example, the therapeutic agent for further administration may include any agents that treats vascular dementia by improving the cognitive function and/or reducing one or more symptoms of vascular dementia in the subject. In one example, the therapeutic agent for further administration may include, but is not limited to, antithrombotic drug, antihypertensive drug, antihyperlipidemic agent, and antidiabetic drug.

In one example, the present disclosure refers to a kit for treating vascular dementia comprising the antibody or antigen-binding fragment thereof disclosed herein and/or instructions for use. In some examples, the reagents provided in the kit as described herein may be provided in separate containers comprising the components independently distributed in one or more containers.

As used in this application, the singular form “a,” “an,” and “the” include plural references unless the context clearly dictates otherwise.

As used herein, the term “at least” when used in the context of a value is understood to mean “greater than or equal to” said value. For example, a sequence having “at least 85% sequence identity” to another sequence would be understood to include a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the other sequence. In another example, an antibody or antigen-binding fragment thereof comprising a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 would be understood to include an antibody or antigen-binding fragment thereof comprising a heavy chain variable region of SEQ ID NO: 7 (i.e. 100% sequence identity), and a light chain variable region of SEQ ID NO: 11 (i.e. 100% sequence identity).

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

The term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies of the present disclosure may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F(ab)₂, scFv (single chain Fv), dAb (single domain antibody), bi-specific antibodies, diabodies and triabodies As used in this application, “antibody fragment” refers to an antibody which has been reduced in length by one or more amino acids while retaining its antigenic activity.

The term “humanized antibody” refers to antibodies from non-human species whose protein sequences have been modified to increase their similarity to antibody variants produced naturally in humans.

The term “antibody variant” as used herein refers to antibodies with differences in their amino acid sequences but still retain their binding specificities to their antigens. For example, one or more conservative amino acid substitutions may be made to the TRPM4 antibody or its antigen-binding fragment thereof while retaining binding specificity to TRPM4.

As used herein “sequence identity” refers to the residues in two sequences that are the same when aligned for maximum correspondence over a specified window of comparison by means of computer programs known in the art such as GAP provided in the GCG program package (Program Manual for the Wisconsin Package, Version 11, 2005, Genetics Computer Group, 575 Science Drive, Madison, Wisconsin, USA 53711) and DNAstar Lasergene.

As used herein the term “treatment” refers to any and all uses which remedy a disease state or symptoms, prevent the establishment of disease, or otherwise prevent, hinder, retard, or reverse the progression of disease or other undesirable symptoms in any way whatsoever.

In the context of this specification, the term “an effective amount” includes within its meaning a non-toxic but sufficient amount of an agent (for example, the antibody or its antigen-binding fragment thereof of the present disclosure) to provide the desired effect. The exact amount required will vary from subject to subject depending on factors such as the species being treated, the age and general condition of the subject, the particular agent (for example, the antibody or its antigen-binding fragment thereof of the present disclosure) being administered and the mode of administration and so forth. Thus, it is not possible to specify an exact “effective amount”. However, for any given case, an appropriate “effective amount” may be determined by one of skill in the art using appropriate means.

In one example, the antibody or its antigen-binding fragment thereof may be administered to a subject in need thereof. Convenient modes of administration include injection (subcutaneous, intravenous, etc.), oral administration, inhalation, transdermal application, topical creams or gels or powders, or rectal administration. Depending on the route of administration, the antibody or its antigen-binding fragment thereof may be coated with a material to protect it from the action of enzymes, acids and other natural conditions which may inactivate its therapeutic activity. The antibody or its antigen-binding fragment thereof may also be administered parenterally or intraperitoneally.

Dispersions of the antibody or its antigen-binding fragment thereof according to the present disclosure may also be prepared in glycerol, liquid polyethylene glycols, and mixtures thereof and in oils. Under ordinary conditions of storage and use, pharmaceutical preparations may contain a preservative to prevent the growth of microorganisms.

The term “pharmaceutical composition” refers to a preparation containing a pharmaceutically active ingredient which is suitable for administration to a subject. The term “pharmaceutically acceptable carrier” refers to an ingredient in a pharmaceutical composition, other than the pharmaceutically active ingredient, which is nontoxic to a subject. The use of such “pharmaceutically acceptable carrier” is well known in the art. A pharmaceutically acceptable carrier includes, but is not limited to, a buffer, excipient, stabilizer, or preservative. The antibody or its antigen-binding fragment thereof may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the antibody or its antigen-binding fragment thereof may be administered by injection. In the case of injectable solutions, the carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, isotonic agents, for example, sugars, polyalcohols such as mannitol, sorbitol, sodium chloride may be included in the pharmaceutical composition. Prolonged absorption of the injectable pharmaceutical compositions can be brought about by including in the pharmaceutical composition an agent which delays absorption, for example, aluminium monostearate and gelatin.

Sterile injectable solutions can be prepared by incorporating the analogue in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Single or multiple administrations of the pharmaceutical compositions according to the present disclosure may be carried out. One skilled in the art would be able, by appropriate means, to determine effective, non-toxic dosage levels of the antibody or its antigen-binding fragment thereof and/or pharmaceutical composition of the present disclosure and an administration pattern which would be suitable for treating the diseases and/or infections to which the antibody or its antigen-binding fragment thereof and pharmaceutical compositions are applicable.

Further, it will be apparent to one of ordinary skill in the art that the optimal course of treatment, such as the number of doses of the antibody or its antigen-binding fragment thereof or pharmaceutical composition of the present disclosure given per day for a defined number of days, can be ascertained using convention course of treatment determination tests.

As used herein, the term “increase” refers to a rise in amount, expression level or number on a positive scale. Conversely, the term “decrease” indicates a change on a negative scale.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the present disclosure embodied therein herein disclosed may be resorted to by those skilled in the art, and that such modifications and variations are considered to be within the scope of this present disclosure.

The disclosure has been described broadly and generically herein. Each of the narrower species and subgeneric groupings falling within the generic disclosure also form part of the present disclosure. This includes the generic description of the present disclosure with a proviso or negative limitation removing any subject matter from the genus, regardless of whether or not the excised material is specifically recited herein.

Other embodiments are within the following claims and non-limiting examples.

### EXAMPLES

### Methods

### Antibody Humanization by CDR Grafting Plus Back Mutation

The structure of parental antibody was modelled by computer-aided homology modelling program. Humanized antibodies were designed using CDR grafting. Briefly, the CDRs of parental antibody were grafted into the human acceptors to obtain humanized light chains and humanized heavy chains for each parental antibody. 4 heavy chains (VH1, VH2, VH3 and VH4) and 4 light chains (VL1, VL2, VL3 and VL4) were paired with each other for affinity ranking experiment.

### Production of Chimeric and Humanized Antibodies

The DNA sequences encoding the chimeric and humanized antibodies heavy and light chains were synthesized and inserted into pcDNA3.4 vector to construct expression plasmids of full-length IgGs. The designed plasmids of heavy and light chain were sent for transfection to HEK 293 or CHO cells using Lipofectamine 2000. The culture media for both HEK 293 and CHO cells was: Dulbecco's Modified Eagle's Medium (DMEM) (12800017; Thermo Fisher Scientific, USA) supplemented with 10% fetal bovine serum (10500064, Thermo scientific); 1.74 g/L sodium bicarbonate; 1.2 g/L HEPES; and 100 U/mL Penicilin-Streptomycin (15140122; thermo scientific). The recombinant IgGs secreted to the medium were purified using protein. The purified antibody was buffer-exchanged into PBS using PD-10 desalting column. The concentration and purity of the purified protein were determined by OD280 and SDS-PAGE, respectively.

### Affinity Ranking of Chimeric and Humanized Antibodies

For affinity ranking, antibodies were captured on the sensor chip through Fc capture method. Peptide-biotin was used as the analyte. The surface was regenerated before the injection of another antibody. The process was repeated until all antibodies were analyzed. The off-rates of antibodies were obtained from fitting the experimental data locally to 1:1 interaction model using the Biacore 8K evaluation software. The antibodies were ranked by their dissociation rate constants (off-rates, kd). Based on the ranking result, the top 3 clones were selected.

### Affinity Measurement of Purified Humanized IgGs

The affinity of purified antibody binding to Peptide-biotin was individually determined using a Surface Plasmon Resonance (SPR) biosensor, Biacore 8K (GE Healthcare). Antibodies were captured on the sensor chip through Fc capture method. Peptide-biotin was used as the analyte. The data of dissociation (kd) and association (ka) rate constants were obtained using Biacore 8K evaluation software. The equilibrium dissociation constants (KD) were calculated from the ratio of kd over ka.

### Western Blot for Hippocampal Neurons or Rat Brain Tissues

The hippocampal neurons or rat brain tissues were homogenized in ice cold radioimmunoprecipitation assay (RIPA) buffer (150 mM NaCl; 1% Igepal CA-630; 0.5% sodium deoxycholate; 0.1% sodium dodecyl sulfate, 50 mM Tris pH8.0; 1 mM EDTA; 1 mM EGTA) containing Mini Protease Inhibitor (Cat #, Roche, Basel, Switzerland) for western blot assay. Protein concentration was determined by Pierce BCA Protein Assay Kit (23227; Thermo Fisher). 80 μg of total protein was resolved on 8% or 15% SDS-PAGE gels at 80 V, and electrophoretically transferred to Immun-Blot PVDF membranes (1620177; Bio-Rad, CA, USA) at 110 V for 2 h at 4° C. After blocking with StartingBlock blocking buffer (37538; Thermo Fisher) containing 0.05% Tween®20 for 1 h at room temperature, membranes were incubated overnight at 4° C. with the following primary antibodies: anti-TRPM4 (ACC-044; Alomone, Jerusalem, Israel) at 1:500 dilution, anti-actin (A1978; Sigma-Aldrich) at 1:5000 dilution and anti-cleaved caspase (9661S; Cell Signaling Technology, MA, USA) at 1:500 dilution. The membranes were then washed and incubated with the respective secondary antibody (A4416; A4914; Sigma-Aldrich) for 1 h at room temperature. Primary and secondary antibodies were both prepared in StartingBlock blocking buffer with 0.05% Tween®20. Protein bands were detected using the Amersham ECL Western Blotting Analysis System (RPN2109, GE Healthcare, IL, USA) and visualized using a medical x-ray processor (MXP-2000; KODAK, NY, USA). ImageJ was used for gel quantification.

### Immunostaining and Immunofluorescence Microscopy

Rats were anesthetized and transcardially perfused with PBS, followed by 4% PFA. The brains were collected and cryosectioned at 30 μm of thickness. After washing twice with 0.1% Triton X-100 in phosphate buffered saline (PBST), the sections were blocked with 10% FBS in PBST for 30 mins at room temperature. The sections were then incubated overnight at 4° C. with primary antibodies diluted with 5% FBS. The primary antibodies used in the study were anti-TRPM4 (ACC-044; Alomone, Jerusalem, Israel). On the following day, the sections were first washed three times with PBST and then incubated for 1 hr at room temperature with the appropriate Alexa Fluor® Dyes secondary antibodies diluted in 5% FBS. The sections were washed three times with PBST and followed by addition of DAPI to stain the nuclei. After washing three times with PBS, the slides were mounted with cover glasses using the FluorSave™ reagent. Antibody extravasation was determined in the PBOCCA rats at 1 day post-surgery by immunoflurescence staining using secondary antibody against rabbit IgG. The results were visualized using the laser scanning confocal microscope system (Fluoview FV31S-SW, Olympus).

### Electrophysiology in Cells

### Whole-Cell Patch Clamp

Whole-cell patch clamp was used to measure TRPM4 currents in primary cultured hippocampal neurons. Patch electrodes were pulled using a Flaming/Brown micropipette puller (P-1000, Sutter Instrument, CA, USA) and polished with a microforge (MF-200, WPI Inc. FL, USA). Whole-cell currents were recorded using a patch clamp amplifier (Multiclamp 700B equipped with Digidata 1440A, Molecular Devices, CA, USA). The bath solution contained (in millimole/liter): NaCl 140, CaCl2) 2, KCl 2, MgCl2 1, glucose 20 and HEPES 20 at pH 7.4. The internal solution contained (in millimole/liter): CsCl 156, MgCl2 1, EGTA 10 and HEPES 10 at pH 7.2 adjusted with CsOH. The cells were pre-treated with rabbit IgG or M4P at a concentration of 20.8 μg/ml in bath solution for 30 mins before recording. Chronic hypoxia was induced by incubating neurons in a hypoxia chamber for 24 h with the presence of IgG or M4P (20.8 μg/ml). Acute hypoxia was induced by applying a bath solution containing 5 mM NaN3 and 10 mM 2-deoxyglucose (2-DG) continuously through a MicroFil (34 Gauge, WPI Inc. USA) around 10 μm away from the recording cells. The flow rate was 200 μl/min. The current-voltage relations were measured by applying voltage ramps for 250 ms from −100 to +100 mV at a holding potential of −70 mV or 0 mV. Membrane potentials were measured by I=0 current-clamp mode when neurons were broken in as whole-cell configuration. Membrane capacitance was recorded by the Membrane Test of Clampex software (Molecular Devices, CA, USA). The sampling rate was set at 20 kHz and filtered at 1 KHz. Data were analyzed using pClamp10, version10.2 (Molecular Devices, CA, USA).

### Calcium Imaging

Hippocampal neurons at 15-18 DIC were loaded with 1 μM Fura-2AM (F1201; Thermo Fisher) for 30 mins in the dark. Cells were then gently washed with PBS, pH7.3 (140 mM NaCl, 5 mM KCl, 1.8 mM CaCl2), 1 mM MgCl2, 10 mM HEPES, 10 mM D-Glucose). On an Olympus IX81 microscope with the MT-20 illumination unit, the cells were continuously superfused with the PBS for around 5 mins before 2 μM glutamate (G1251; Sigma-Aldrich) or 100 μM NMDA (ab120052; Abeam, Cambridge, UK) was added for 30 seconds. Fura-2 ratio images were taken at alternating excitation wavelengths of 340 nm and 380 nm every 0.4 s using an Olympus Inverted Microscope IX81 fitted with an MT20 Illumination System (Olympus). Intracellular calcium concentration was calculated from the ratio of Fura-2 dye wavelengths using the Olympus Xcellence RT software.

### In Vivo Electrophysiological Recording

The rats were anesthetized with urethane purchased from Sigma Aldrich, USA (2 g/kg given as four i.p. injections of 0.5 g/kg each, every 20 min; further supplements of 0.5 g/kg were administered when necessary). The fur on the top of the head was shaved, and local anesthetic xylocaine (5 mg/kg) was administered subcutaneously to the skin and tissue along the incision line over the skull. The body temperature was maintained at 37.0±0.5° C. The skull was exposed, and burr holes were drilled based on standard stereotaxic measurements relative to bregma. A monopolar recording electrode was placed in the CA1 (AP: −4.2 mm, ML: −3.0 mm, V: −3.0 mm) to record the extracellular field excitatory postsynaptic potentials (fEPSPs). Signals were amplified, filtered and digitized at 10 kHz by an A/D converter (PowerLab system, ADInstruments) and stored in a computer using LabChart 7 program software for subsequent offline analysis. A bipolar concentric stimulating electrode was placed in the contralateral CA3 region of the hippocampus (AP: −4.2 mm, ML: +3.0 mm, V: −4.0 mm) and connected to a stimulus isolator unit (ML180 Stimulus isolator, AD Instruments) providing a constant current output. Screws were placed in the bone overlying the frontal cortex to serve as references and ground connections for the recording electrodes. The intensity required to elicit the maximal fEPSP amplitude was determined via input-output curves with 0.2 ms stimulation pulses delivered to the CA3 with ascending series of stimulus intensities between 0.1 and 1.0 mA (in 0.1 mA increments). From the input-output curves, a stimulation intensity eliciting approximately 50-60% of the maximal fEPSP amplitude was then used for the remainder of the experiment. For each experiment, a 60 min period of initial baseline recorded every 30 s was obtained to achieve a stable baseline. Subsequently, single train of theta burst stimulation (TBS) was delivered to the CA3, consisting of five pulses (at 100 Hz) per burst, with burst repeated at 200 ms interval for a total of ten bursts. Finally, the fEPSPs were recorded every 30 s for 3 h following TBS. Data obtained from inaccurate placement of electrodes were excluded from the analysis.

### Induction of Head Injury in Animal Model

Wistar rats (InVivos, Singapore) weighing approximately 65-85 g were subjected to Traumatic Brain Injury (TBI). The animals were anesthesized with ketamine (75 mg/kg) and xylazine (10 mg/kg) intraperitoneally. The device consists of a unique column of an acrylic tube with a freely falling 15.6 mm diameter brass weight weighing 150 g by gravity onto the head. The brass weight falls through a 1-m vertical section of the acrylic tube held in place with ring a stand. A 4-mm thick steel disc was fixed to the left portion of the rat head to restrict the zone of contact to the left top of the rat head. The anesthetized rats were placed in a prone position on a foam bed and a stage consisting of aluminium foil to hold subjects in place. The weight was tethered to the instrument with commercially available fishing line to ensure that the weight did not travel further than 1 cm beyond the dorsal surface of the head and could not produce “re-hit” to the head or body of the rat. Immediately after impact, topical lidocaine was applied to the heads of the rats and they were placed in a clean cage. Animals experiencing a sham injury were similarly anesthetized, underwent similar procedures with no weight drop.

### Real Time PCR

Hippocampal neurons and brain tissues were first homogenized with ice cold TRIzol™ Reagent, followed by total RNA extraction using the Direct-zol™ RNA MiniPrep kit (Zymo Research, CA, USA) and quantification using a NanoDrop Spectrophotometer (Thermo Fisher). First strand cDNA was synthesized from the total RNA using an 18-mer oligodT with the SuperScript™ III First-Strand Synthesis System (Thermo Fisher). Using a LightCycler 96 Instrument (Roche), RT-PCR was performed in the FastStart Essential DNA Green Master mix (06402712001; Roche) to detect expression levels of TRPM4. The primer sequences were SEQ ID NO: 64 and SEQ ID NO: 65 as shown below. The RT-PCR consists of an initial denaturation step at 95° C. for 15 mins, followed by 35 cycles of denaturation step at 95° C. for 15 s, annealing step at 60° C. for 30 s, and extension step at 72° C. for 30 s. A final extension step at 72° C. for 5 mins was also performed.

Primers used for the detection of the expression levels of TRPM4:

(SEQ ID NO: 64)
AGAGAAATACACGGAGCCCCA (forward primer);
(SEQ ID NO: 65)
CCCACACTGGCACAAGGTC (reverse primer).

### Beam Walking and Tail Suspension Assays

### Beam Walking

Beam walking was used to examine the balance and motor coordination of the post-TBI rats. The rat's home cage was placed at one end of the diameter long round beam, and the start point was at another end of the beam. One day before the induction of TBI, the rats were given four trials to learn to walk from the start of the beam to home cages. No video was recorded at this time. Once the rat reached its home cage, it was permitted to remain in the cage for 60 sec to reinforce the target location. For post-TBI, the video camera was set to record from the starting point of the beam, and positioned to view down the length of the beam. The rat was given 3 video tapped trials, each separated by the 30 sec reinforcement. A researcher blinded to the experimental groups scored the videos for hind leg foot slips off the round beam.

### Tail Suspension Test

Tail suspension test is widely used for assessing depressant-like activity in animals. In this test, rats were suspended above the floor by adhesive tape placed approximately 1 cm from the tip of the tail. Rats were considered immobile only when they hung passively and completely motionless. The total duration of immobility induced by tail suspension was recorded during a 6-min period.

### Occlusion of Bilateral Common Carotid Artery in Animal Model

All animal experiments were performed in accordance to the approved guidelines of the Institutional Animal Care and Use Committee of the National Neuroscience Institute, Singapore and the Universiti Sains Malaysia, Malaysia. Male Sprague Dawley (SD) rats, weighing between 250 to 300 g, were anesthetized with ketamine (75 mg/kg) and xylazine (10 mg/kg) intraperitoneally. Both common carotid arteries were exposed and ligated with a 5/0 silk suture. The sham-operated rats underwent the similar procedure except for the ligation. After operation, a single dose of 100 μg M4P or control rabbit IgG (I5006; Sigma-Aldrich, MI, USA) was injected intravenously via tail vein. Both control rabbit IgG and M4P were prepared in an IgG elution buffer (21004, Thermo Fisher Scientific).

### Morris Water Maze Experiment

One day before the start of training, each rat was given a pretraining session in which they were allowed to swim freely in a water pool (160 cm diameter, with a depth of 70 cm of water) for 60 s without an escape platform. The pool was placed in a large test room, and surrounded by various visual cues. During training, the pool was filled to a depth of 39 cm with water maintained at 25±1° C. and made opaque by adding white paint. A platform (10 cm diameter) was situated 2 cm below water surface. The pool was divided into four quadrants, with the platform fixed at one quadrant. Daily training consists of four trials in which the rat was placed in the water from four different starting points and the latency of escaping onto the platform was recorded. This was conducted for five consecutive days. A maximum of 60 s were allowed for each trial during which the rat had to find the platform and climb onto it. If the rat failed to find the platform within 60 s, the trial was terminated and a maximum score of 60 s was assigned. The rat was then guided to the hidden platform by hand and allowed to stay on the platform for 10 s before it was removed from the water. On the sixth day, each rat was subjected to a probe trial in which there was no platform placed in the pool. The time of crossing the former platform quadrant and the total time of crossing all quadrants were recorded for 1 min. The percentage of crossing the quadrant of the former platform represented the ratio between the time of crossing the quadrant of the former platform and the total time of crossing all quadrants. The percentage was calculated as a measure of spatial reference memory.

### Dynamic Light Scattering (DLS)

Antibodies were dissolved using a phosphate buffered saline to make a concentration of 3 mg/ml. The samples were then loaded into capillaries. Thermal ramps were run from 25° C. to 70° C. in 1° C. increment. Measurement was performed using a DLS system (Wyatt, DynaPro Plate Reader III).

### Non-Reducing Capillary Electrophoresis-Sodium Dodecyl Sulfate (CE-SDS-NR)

Antibodies were dissolved using a phosphate buffered saline to make a concentration of 3 mg/ml. For high temperature test, the samples were incubated at 40° C. for 7, 14, and 28 days. For low pH stability test, the solution pH was adjusted to pH3.5, and the samples were incubated for 2 and 4 hours at 37° C. After incubation, 100 g of all samples was obtained to prepare the loading sample solution by adding the sample buffer (phosphate salt, citric acid and SDS), followed by the addition of 10 kD Internal Standard marker and alkylating reagent-NEM (N-Ethylmaleimide). The samples were then incubated at 70° C. for 10 min and allowed to cool down to room temperature. The resulting solutions were centrifuged, and the supernatants were taken for analysis on the CE system (SCIEX, P/ACE MDQ plus).

### Results

### TRPM4 Blocking Antibody Improved Functional Recovery Following Traumatic Brain Injury (TBI)

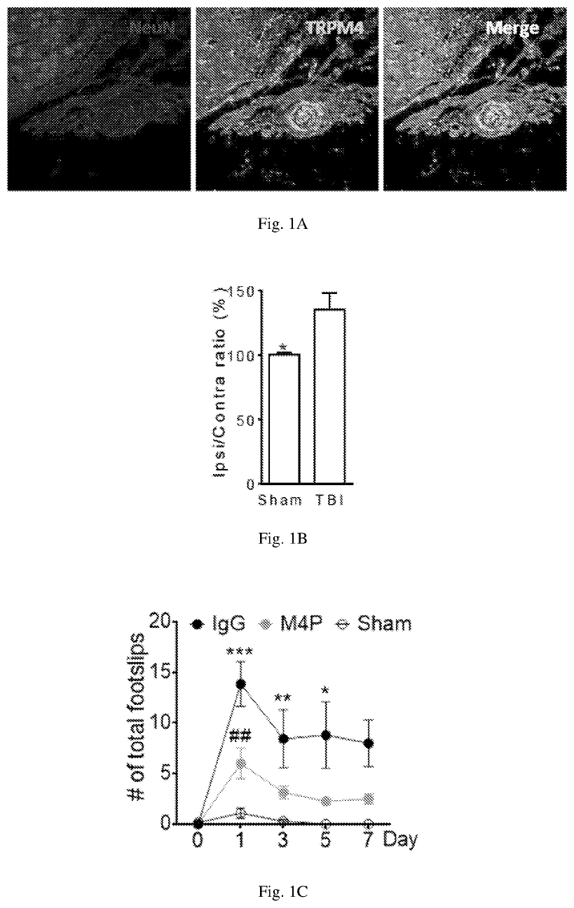

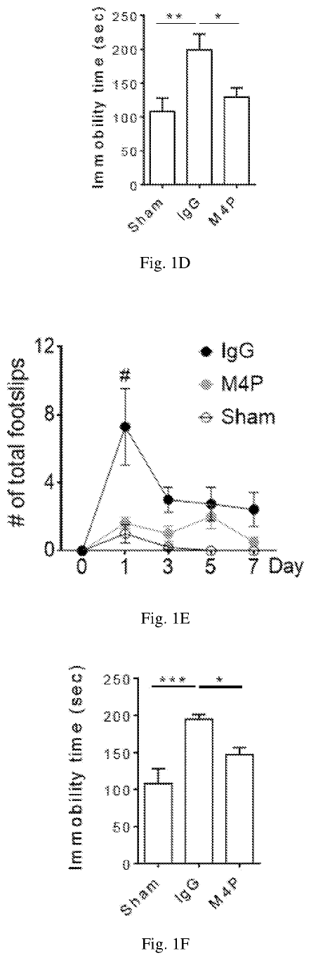

To examine the effect of TRPM4 in TBI, a weight drop experiment was performed to induce head injury in rats. One day after TBI induction, the protein TRPM4 was found to be expressed mainly at cerebral vasculature (FIG. 1A), but not in neurons labeled with a neuron-specific protein, NeuN (a neuronal marker). Real time PCR verified the upregulation of the expression TRPM4 mRNA (FIG. 1B). In healthy brain, TRPM4 expression is low in endothelial cells. TRPM4 blocking antibody M4P or control IgG was administered intravenously after the induction of TBI, and compared with sham operated animals (i.e., animals which were subjected to sham surgery). In TBI animals with skull fracture and severe symptoms, M4P treatment significantly reduced functional loss as assessed by the beam walking (FIG. 1C) and tail suspension assays (FIG. 1D). In TBI animals without skull fracture and mild symptoms, M4P treatment again alleviated functional loss in the beam walking (FIG. 1E) and tail suspension assays (FIG. 1F).

### TRPM4 Blocking Antibody Reduced Chronic Hypoxia Induced Neuronal Excitotoxicity

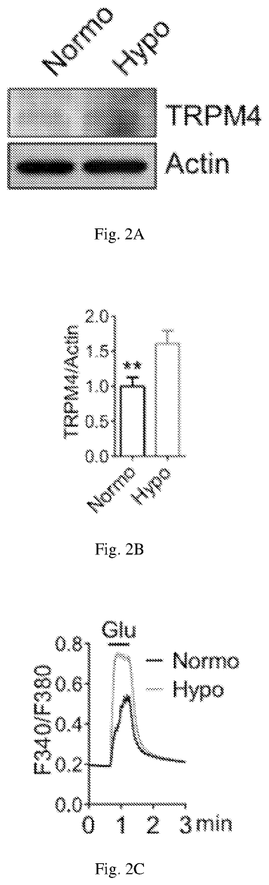

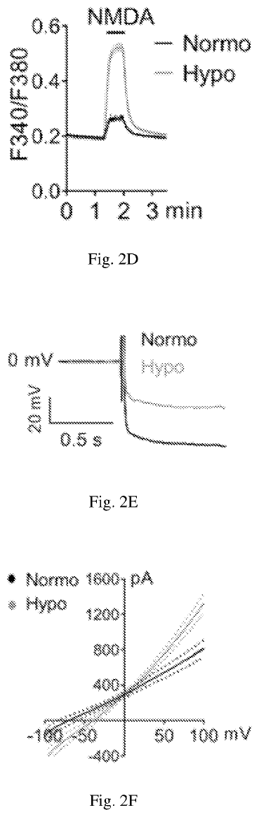

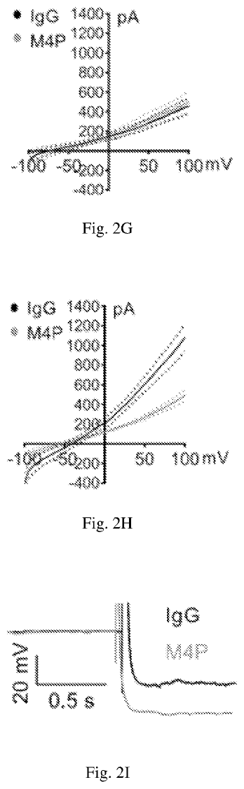

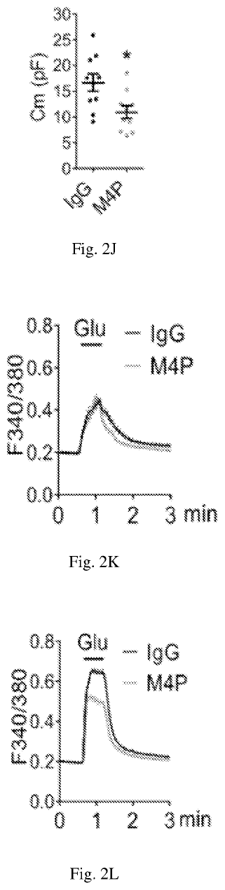

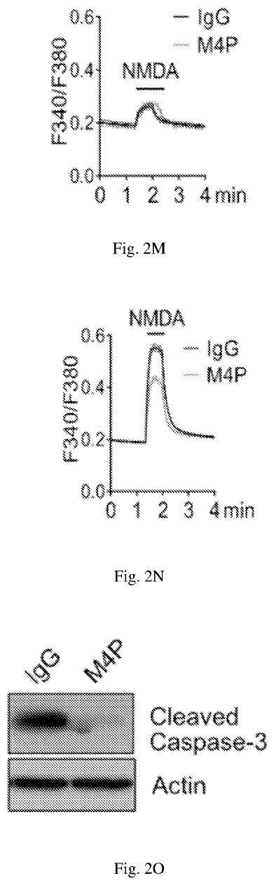

In primary cultured neurons, TRPM4 protein expression in cultured hippocampal neurons was upregulated after exposure to chronic hypoxia (FIGS. 2A and 2B). In chronic hypoxic neurons, application of glutamate (FIG. 2C) and NMDA (FIG. 2D) greatly enhanced calcium entry into the neurons, a phenomenon known as excitotoxicity. Characterized by a calcium over-influx via NMDA receptors, excitotoxicity is a major mechanism for neuronal cell death during hypoxia. Hypoxia could depolarize membrane potential (FIG. 2E) and remove magnesium blockade to NMDA receptors. During chronic hypoxia, the permeability of currents across membrane were found to be enhanced (FIG. 2F). Under normoxic condition, incubation of TRPM4 blocking antibody M4P did not change membrane permeability as compared to control IgG treatment (FIG. 2G). In sharp contrast, transmembrane currents were greatly suppressed by M4P under hypoxic conditions (FIG. 2H). TRPM4 mainly conducts monovalent cation. Blocking TRPM4 thus caused membrane repolarization during hypoxia (FIG. 2I), suggesting that inhibiting sodium influx via TRPM4 could repolarize membrane potential. As sodium influx via activated TRPM4 during hypoxia could increase cell volume, a volume reduction of the neuronal cell upon M4P treatment was observed (FIG. 2J). Next, the effect of M4P on calcium influx via NMDA receptors was examined. Under normoxic conditions, there was no difference between M4P and control IgG after treatment with glutamate (FIG. 2L) or NMDA (FIG. 2M). However, under chronic hypoxia, M4P greatly reduced calcium influx after treatment with glutamate (FIG. 2L) or NMDA (FIG. 2N). With the suppression of excitotoxicity, a reduction of apoptosis was observed (FIG. 2O). This experiment verified the two mechanisms of neuroprotection from TRPM4 blocking antibody: 1) blocking sodium influx could reduce hypoxia induced oncotic cell death and 2) TRPM4 antibody could reduce calcium influx via NMDA receptor during chronic hypoxia. The most likely mechanism is that M4P could repolarize membrane potential of hypoxic neurons, and maintain the magnesium-mediated NMDA blockade.

### TRPM4 Blocking Antibody Reduced Apoptosis and Improved Cognition in Animal Model of Vascular Dementia

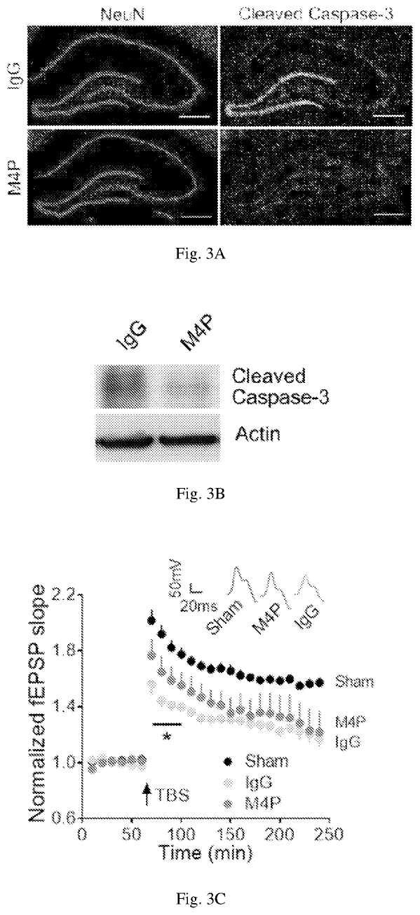

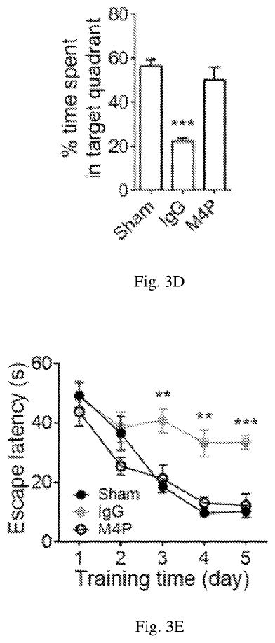

The effect of M4P in a rat model of vascular dementia was examined by occluding bilateral common carotid artery (BCCAO). In this model, chronic hypoxia damaged hippocampal neurons and impaired cognition. After occlusion, control IgG or M4P was delivered into the rats intravenously. One month after treatment, apoptosis was significantly observed in the hippocampal neurons of the IgG-treated animals. On the other hand, M4P greatly reduced hippocampal apoptosis (FIGS. 3A and 3B). When the long-term potentiation (LTP) of Schaffer collateral synapses in CA1 pyramidal cells was measured, theta-burst stimulation (TBS) resulted in a robust increase of field excitatory postsynaptic potential (fEPSP) amplitude in all 3 groups. Compared to IgG, M4P treatment greatly enhanced fEPSP (FIG. 3C). In the Morris water maze experiment, M4P treatment significantly shortened the escape latency in BCCAO rats over the five training days (FIG. 3E). At the end of the place task, M4P group had a similar performance as sham-operated group, indicating a better spatial memory retention (FIG. 3D). These results suggested that M4P could enhance learning and memory in animal model of vascular dementia by protecting hippocampal neurons and improving synaptic plasticity.

### Validation of Antibody Binding Affinity of Humanized Antibodies Against TRPM4

Humanized antibodies were generated by replacing the framework regions (FRs) from mouse M4M antibody with counterparts from human, while maintaining the antibody's complementarity-determining regions (CDRs) binding regions. The antibody binding affinity of the chimeric and humanized antibodies was assessed by surface plasmon resonance (SPR) as shown in Table 1. Antibody clone A1 is the chimeric antibody and antibody clones A2-7 are humanized antibodies. Based on the results obtained from SPR, humanized clone A2 has the strongest SPR signal. The antigen binding kinetics to the chimeric and humanized antibodies are shown in Table 2. For affinity ranking, antibodies were captured on the sensor chip through Fc capture method. Peptide-biotin was used as the analyte. The surface was regenerated before the injection of another antibody. The process was repeated until all antibodies are analyzed. The off-rates of antibodies were obtained from fitting the experimental data locally to 1:1 interaction model using the Biacore 8K evaluation software. The antibodies were ranked by their dissociation rate constants (off-rates, kd). Based on the ranking result, the top humanized clones were selected. Table 3 shows the humanization design and back mutation sites in the framework regions of the heavy and light chain regions of the humanized antibody clones. Bolded and underlined amino acids correspond to the back mutation sites in the framework regions.

Humanized antibody clones (A2-A7) were generated by combining various heavy and light chain variable domains (VH1-VH4 variable regions and VL1-VL4 variable regions). The amino acid sequence of the variable domains for heavy and light chains of the humanized antibodies are as follows:

VH1 variable region:
(SEQ ID NO: 7)
QVQLQESGPGLVKPSQTLSLTCTVSGYSITSDYAWNWIRQHPGKGLEW
IGYLSYSGVTSYNPSLKGRVTISVDTSKNQFSLKLSSVTAADTAVYYC
ARKGTYYRYEGSYWYFDVWGQGTTVTVSS
VH2 variable region:
(SEQ ID NO: 8)
QVQLQESGPGLVKPSQTLSLTCTVSGYSITSDYAWNWIRQHPGKGLEW
MGYLSYSGVTSYNPSLKGRVTISRDTSKNQFSLKLSSVTAADTAVYYC
ARKGTYYRYEGSYWYFDVWGQGTTVTVSS
VL1 variable region:
(SEQ ID NO: 11)
DIQMTQSPSSLSASVGDRVTITCHANQNIDVWLSWYQQKPGKAPKLLI
YKTSNLHTGVPSRFSGSGSGTDFTFTISSLQPEDIATYYCQQGQSYPL
TFGGGTKLEIK
VL2 variable region:
(SEQ ID NO: 12)
DIQMTQSPSSLSASVGDRVTITCHANQNIDVWLSWYQQKPGKAPKLLI
YKTSNLHTGVPSRFSGSGSGTDFTLTISSLQPEDIATYYCQQGQSYPL
TFGGGTKLEIK
VL3 variable region:
(SEQ ID NO: 13)
DIQMTQSPSSLSASVGDRVTITCHANQNIDVWLSWYQQKPGNAPKLLI
YKTSNLHTGVPSRFSGSGSGTDFTFTISSLQPEDIATYYCQQGQSYPL
TFGGGTKLEIK
VL4 variable region:
(SEQ ID NO: 14)
DIQMTQSPSSLSASVGDRVTITCHANQNIDVWLSWYQQKPGNAPKLLI
YKTSNLHTGVPSRFSGSGSGTDFTLTISSLQPEDIATYYCQQGQSYPL
TFGGGTKLEIK

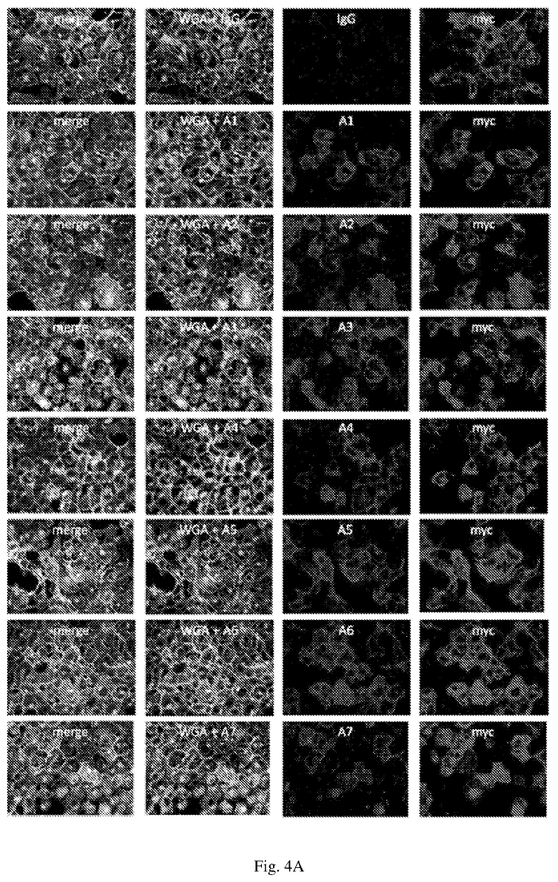

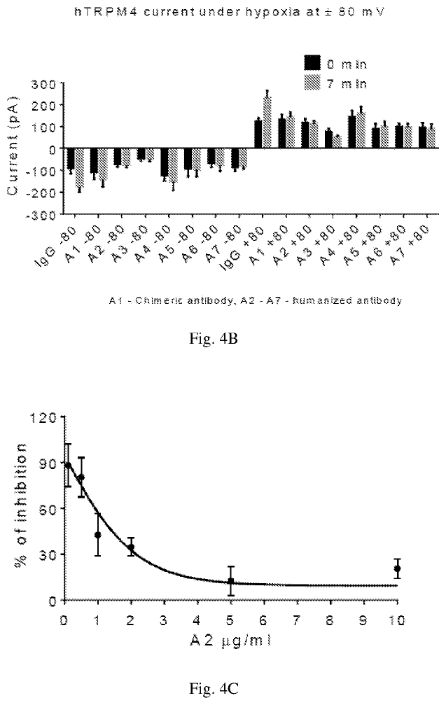

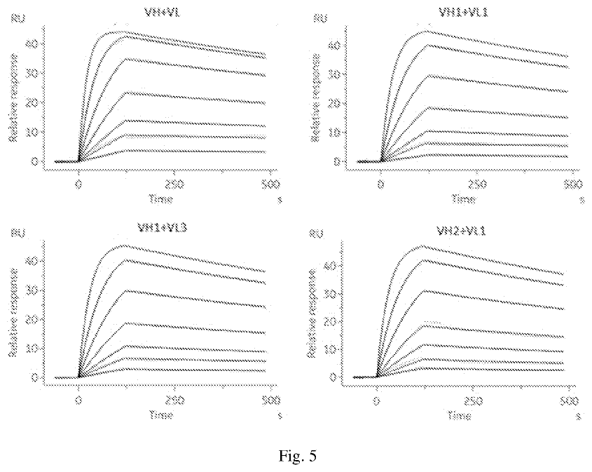

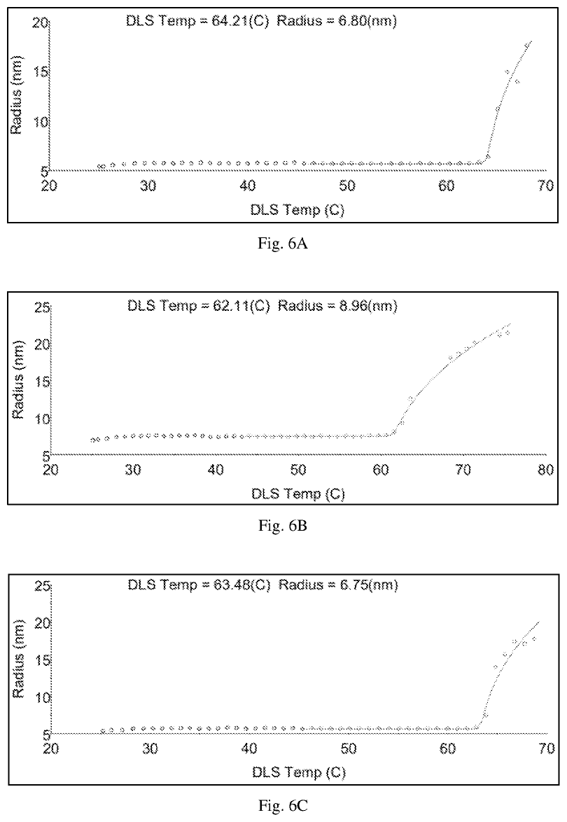

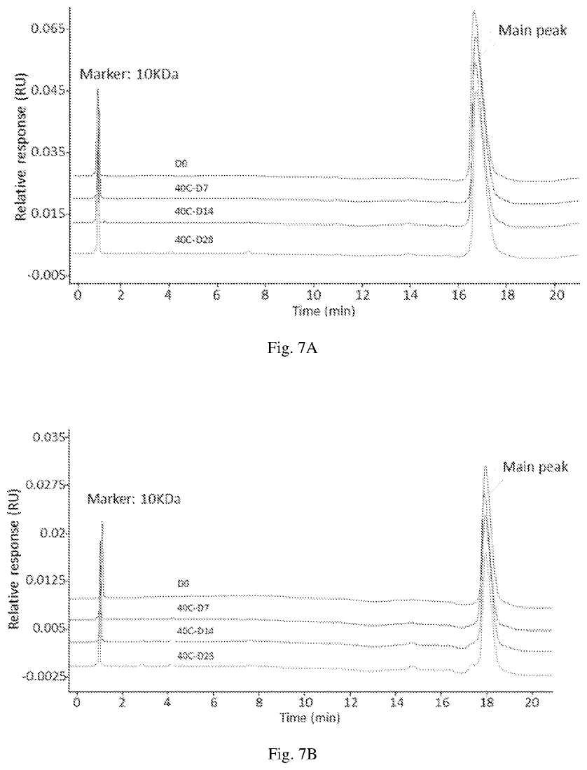

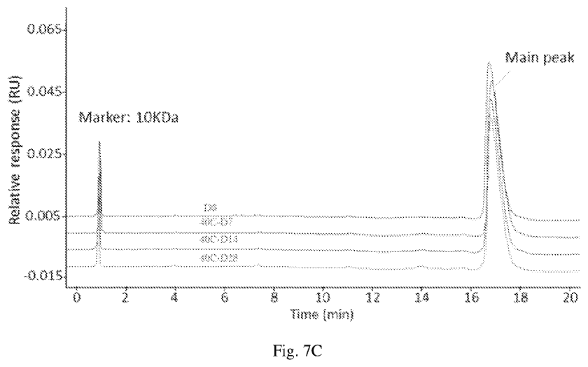

Biochemical and functional characterization were performed to evaluate the antigenic properties of the humanized antibodies. Immunofluorescent staining showed that all six humanized antibodies A2-7 and the chimeric antibody A1 could stain human TRPM4 in transfected HEK 293 cells (FIG. 4A). In addition to staining cytosolic TRPM4, colocalization with surface marker WGA suggested that these antibodies also recognized surface TRPM4. Next, functions of the humanized antibodies were evaluated by electrophysiology (FIGS. 4B and 4C). Human TRPM4 current was significantly elevated by a 7-minute hypoxia treatment in control IgG treated cells (FIG. 4B). All 6 humanized antibodies (A2-A7) and the chimeric antibody A1 successfully inhibited hypoxia-induced current increase at a concentration of 20 μg/ml. Since humanized antibody A2 has the least number of back mutation sites while maintaining excellent binding affinity, the dose-dependent effect of A2 on hypoxia-induced TRPM4 current increase was further determined (FIG. 4C). Using human IgG as a control, the IC₅₀ of A2 was determined to be 1.23 μg/ml in a dose-dependent inhibition assay. No difference was be found when the dose was increased above 5 μg/ml (FIG. 4C). In comparison with the dose of 20.8 μg/ml that was required for M4M or M4P to inhibit hypoxia-induced current increase, the results indicated that humanized antibody clone A2 has better sensitivity in blocking TRPM4 compared to its mouse or rabbit antibody counterpart. The binding affinity and kinetics of different humanized antibodies (A1, A2, A4 and A6) to Peptide-biotin were also shown in the form of sensor-grams (FIG. 7). The overlaid smooth black lines of the sensor-grams represent the kinetic fit of the binding response signals at different antibody concentrations to a 1:1 interaction model.

TABLE 1
Binding kinetics of antibody to antigen

| Ligand | Analyte | Chi² (RU²) | ka (1/Ms) | kd (1/s) | KD (M) | Rmax (RU) |
|---|---|---|---|---|---|---|
| NC | peptide-biotin | NA | NA | NA | NA | NA |
| VH + VL (A1) | peptide-biotin | 1.61E−02 | 3.25E+05 | 1.50E−03 | 4.61E−09 | 24.3 |
| VH1 + VL1 (A2) | peptide-biotin | 2.24E−02 | 2.49E+05 | 1.66E−03 | 6.66E−09 | 28.2 |
| VH1 + VL2 (A3) | peptide-biotin | 2.66E−01 | 3.27E+05 | 3.21E−03 | 9.81E−09 | 25.4 |
| VH1 + VL3 (A4) | peptide-biotin | 4.42E−02 | 2.52E+05 | 1.77E−03 | 7.00E−09 | 26.9 |
| VH1 + VL4 (A5) | peptide-biotin | 8.68E−02 | 2.17E+05 | 2.17E−03 | 9.96E−09 | 24 |
| VH2 + VL1 (A6) | peptide-biotin | 5.72E−02 | 1.84E+05 | 1.95E−03 | 1.06E−08 | 31.1 |
| VH2 + VL2 (A7) | peptide-biotin | 9.44E−02 | 2.56E+05 | 2.95E−03 | 1.15E−08 | 29.6 |
| VH2 + VL3 | peptide-biotin | 3.32E−02 | 2.03E+05 | 2.19E−03 | 1.08E−08 | 26.7 |
| VH2 + VL4 | peptide-biotin | 2.49E−02 | 2.15E+05 | 1.85E−03 | 8.62E−09 | 29.2 |
| VH3 + VL1 | peptide-biotin | 3.00E−02 | 3.42E+05 | 1.49E−03 | 4.36E−09 | 28.2 |
| VH3 + VL2 | peptide-biotin | 7.68E−02 | 4.38E+05 | 1.98E−03 | 4.53E−09 | 26.3 |
| VH3 + VL3 | peptide-biotin | 7.77E−02 | 4.09E+05 | 1.80E−03 | 4.40E−09 | 29.5 |
| VH3 + VL4 | peptide-biotin | 1.42E−01 | 6.08E+05 | 1.35E−03 | 2.22E−09 | 26.8 |
| VH4 + VL1 | peptide-biotin | 2.76E−02 | 3.05E+05 | 1.36E−03 | 4.47E−09 | 32.7 |
| VH4 + VL2 | peptide-biotin | 7.55E−02 | 3.03E+05 | 1.32E−03 | 4.37E−09 | 30.3 |
| VH4 + VL3 | peptide-biotin | 3.93E−02 | 3.32E+05 | 1.43E−03 | 4.32E−09 | 28.3 |
| VH4 + VL4 | peptide-biotin | 2.15E−01 | 4.54E+05 | 2.01E−03 | 4.43E−09 | 54.4 |

TABLE 2
Binding kinetics of antigen to antibodies

| Ligand | Analyte | Chi² (RU²) | ka (1/Ms) | kd (1/s) | KD (M) | Rmax (RU) |
|---|---|---|---|---|---|---|
| VH + VL (A1) | peptide-biotin | 1.24E−01 | 3.12E+05 | 5.43E−04 | 1.74E−09 | 44.2 |
| VH1 + VL1 (A2) | peptide-biotin | 1.04E−01 | 1.91E+05 | 6.05E−04 | 3.16E−09 | 45.8 |
| VH1 + VL3 (A4) | peptide-biotin | 7.35E−02 | 1.95E+05 | 6.20E−04 | 3.18E−09 | 46.1 |
| VH2 + VL1 (A6) | peptide-biotin | 1.62E−01 | 1.65E+05 | 6.62E−04 | 4.02E−09 | 48.3 |

TABLE 3
Humanization design and back mutation sites

| Germline | Chain Type | FR1 | CDR1 | FR2 | CDR2 |
|---|---|---|---|---|---|
| | VH | QVQLQESGPGLVKPSQSLSLTCTVT | GYSITSDYAWN | WIRQFPGNKLQWMG | YLSYSGVTSYNPSLKG |
| 72.4% | VH1 | QVQLQESGPGLVKPSQTLSLTCTVS | GYSITSDYAWN | WIRQHPGKGLEWIG | YLSYSGVTSYNPSLKG |
| IGHV4- | | | | | |
| 31*02 | | | | | |
| | VH2 | QVQLQESGPGLVKPSQTLSLTCTVS | GYSIFSDYAWN | WIRQMPGKGLEWMG | YLSYSGVTSYNPSLKG |
| | VH3 | QVQLQESGPGLVKPSQTLSLTCTVS | QYSITSDYAWN | WIRQHPGKGLQWMG | YLSYSCVTSYNPSLKG |
| | VH4 | QVQLQESGPGLVKPSQTLSLTCTVS | GYSITSDYAWN | WIRQHPGKGLQWMG | YLSYSGVTSYNPSLKG |
| | VL | [symbol] | HANQNIDVWLS | WYQQKPGNVPKLLIY | KTSNLHT |
| 77.9% | VL1 | DIQMTQSPSSLSASVGDRVTITC | HANQNIDVWLS | WYQQKPGKAPKLLIY | KTSNLHT |
| IGKV1- | | | | | |
| 33*01 | | | | | |
| | VL2 | DIQMTQSPSSLSASVGDRVTITC | HANQNIDVWLS | WYQQKPGKAPKLLIY | KTSNLHT |
| | VL3 | DIQMTQSPSSLSASVGDRVTITC | HANQNIDVWLS | WYQQKPGNAPKLLIY | KTSNLHT |
| | VL4 | DIQMTQSPSSLSASVGDRVTITC | HANQNIDVWLS | WYQQKPGNAPKLLIY | KTSNLHT |

| Germline | Chain Type | FR3 | CDR3 | FR4 |
|---|---|---|---|---|
| | VH | [symbol] | KGTYYRVEGSYWYFDV | WGAGTTVTVSS |
| 72.4% | VH1 | RVTISVDTSKNQFSLKLSSVTAADTAVYYCAR | KGTYYRYEGSYWYFDV | WGQGTTVTVSS |
| IGHV4- | | | | |
| 31*02 | | | | |
| | VH2 | RVTISRDTSKNQFSLKLSSVTAADTAVYYCAR | KGTYYRYEGSYWYFDV | WGQGTTVTVSS |
| | VH3 | RITISRDTSKNQFSLKLSSVTAADTAVYYCAR | KGTYYRYEGSYWYFDV | WGQGTTVTVSS |
| | VH4 | RITISRDTSKNQFFLKLSSVTAADTAVYYCAR | KGTYYRYEGSYWYFDV | WGQGTTVTVSS |
| | VL | GVPSRFSGSGSGTDFTLTISSLQPEDIATYYC | QQGQSYPLT | FGGGTKLEIK |
| 77.9% | VL1 | GVPSRFSGSGSGTDFTFTISSLQPEDNATYYC | QQGQSYPLT | FGQGTKLEIK |
| IGKV1- | | | | |
| 33*01 | | | | |
| | VL2 | GVPSRFSGSGSGTDFTLTISSLQPEDIATYYC | QQGQSYPLT | FGQGTKLEIK |
| | VL3 | GVPSRFSGSGSGTDFTFTISSLQPEDIATYYC | QQGQSYPLT | FGQGTKLEIK |
| | VL4 | GVPSRFSGSGSGTDFTLTISSLQPEDIATYYC | QQGQSYPLT | FGQGTKLEIK |

[symbol]

indicates data missing or illegible when filed

### Stability of Humanized Antibodies

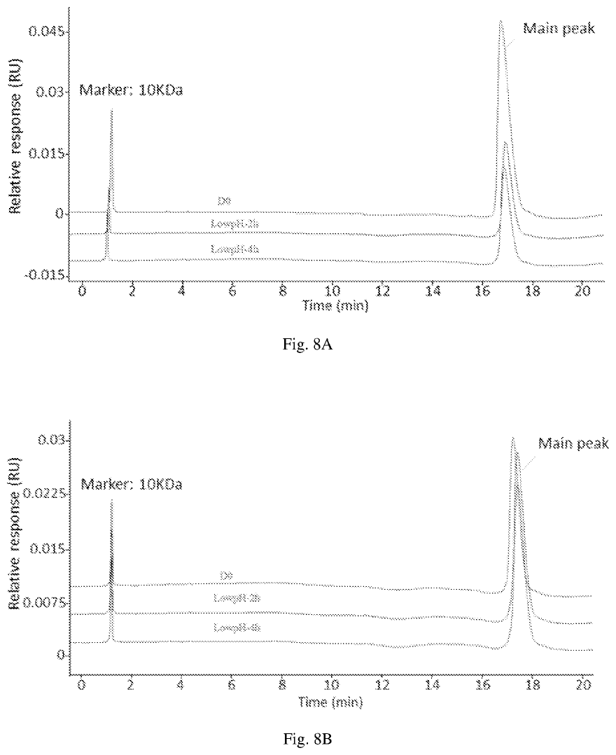

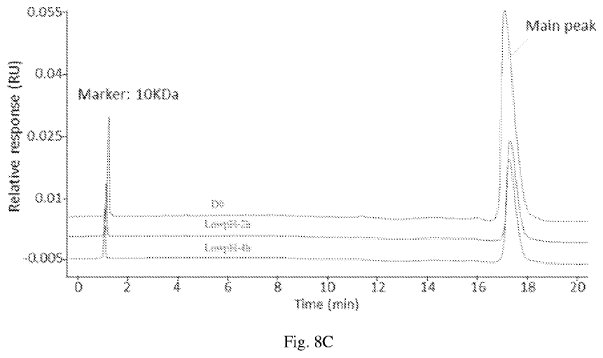

To determine the stability of the humanized antibodies, antibody clones A2, A4 and A6 were incubated under high temperature and low pH. The integrity of the antibody clones was then examined. All three antibody clones demonstrated well tolerance for high temperature (FIGS. 8A, 8B and 8C) and low pH (FIGS. 10A, 10B and 10C). The antibody clones were stable at above 60° C. (FIGS. 8A, 8B and 8C) and they demonstrated no signs of degradation under pH3.5 for up to 4 hours (FIGS. 10A, 10B and 10C). Antibody clones A2, A4 and A6 were also incubated at 40° C. for 28 days and all three antibody clones were stable after incubating for 28 days (FIGS. 9A, 9B and 9C). The high stability means that the humanized antibodies can maintain an active form in the body for a long time.

### DISCUSSION

The present disclosure describes humanized antibodies specific against TRPM4 and their use in treating TBI and vascular dementia. The humanized antibodies disclosed herein effectively inhibits TRPM4 activity and has a therapeutic potential for TBI and vascular dementia. The present disclosure has also shown the protective effect of TRPM4 specific blocking antibodies in TBI and vascular dementia.

In TBI (or head injury), the presence of cerebral edema shortly after brain damage remains a significant challenge for disease management. Cerebral edema is caused by the loss of vascular integrity, always leading to severe permanent functional loss or even death. In the present disclosure, TRPM4 was found to be upregulated in vasculature in head injury. Upregulation and activation of TRPM4 has been proved to cause direct vascular damage. Blocking TRPM4 thus helps maintain the integrity of blood brain barrier (BBB). Therefore, the functional improvement arising from using TRPM4 blocking antibody is likely to be achieved through protecting vasculature after head injury.

As discussed above, chronic hypoxia is associated with vascular dementia, and chronic excitotoxicity has been postulated to contribute to the progression of vascular dementia. TRPM4 blocking antibody is a therapeutic option to control excitotoxicity in brain disorders with chronic hypoxia. There exist two mechanisms for the neuroprotection of TRPM4 antibody: the first is to alleviate oncotic cell death; and the second is to repolarize membrane potential and reduce calcium influx via NMDA receptors. In healthy brains, antibody is unlikely to pass through the intact BBB. However, BBB breakdown has been identified in vascular dementia. In animal model of BCCAO, BBB integrity was also found to be compromised. Therefore, TRPM4 blocking antibody can gain access to the hypoxic neurons via the disrupted BBB.

The present disclosure describes for the first time the therapeutic use of humanized antibodies specific to TRPM4 in treating TBI and vascular dementia. The humanized antibodies of the present disclosure have the following advantages:
1. The humanized antibodies of the present disclosure are expected to demonstrate enhanced therapeutic effects especially in human patients due to lower risk of immune rejection compared to using existing rabbit or mouse antibodies specific against TRPM4.
2. The effective dosage of the humanized antibody clones required to inhibit TRPM4 activity in vitro is about 17 times lower than the effective dosage required for the mouse or rabbit antibody counterpart.
3. The humanized antibody clones comprise unique amino acid sequences of the variable domains for the heavy and light chains.

SEQUENCE LISTING

| SEQ ID NO | Sequence Name | Sequence |
|---|---|---|
| 1 | heavy chain CDR1 | GYSITSDYAWN |
| 2 | heavy chain CDR2 | YLSYSGVTSYNPSLKG |
| 3 | heavy chain CDR3 | KGTYYRYEGSYWYFDV |
| 4 | Light chain CDR1 | HANQNIDVWLS |
| 5 | Light chain CDR2 | KTSNLHT |
| 6 | Light chain CDR3 | QQGQSYPLT |
| 7 | VH1 variable region | QVQLQESGPGLVKPSQTLSLTCTVSGYSITSDYAWNWIRQHPG |
| | | KGLEWIGYLSYSGVTSYNPSLKGRVTISVDTSKNQFSLKLSSVT |
| | | AADTAVYYCARKGTYYRYEGSYWYFDVWGQGTTVTVSS |
| 8 | VH2 variable region | QVQLQESGPGLVKPSQTLSLTCTVSGYSITSDYAWNWIRQHPG |
| | | KGLEWMGYLSYSGVTSYNPSLKGRVTISRDTSKNQFSLKLSSV |
| | | TAADTAVYYCARKGTYYRYEGSYWYFDVWGQGTTVTVSS |
| 9 | VH3 variable region | QVQLQESGPGLVKPSQTLSLTCTVSGYSITSDYAWNWIRQHPG |
| | | KGLQWMGYLSYSGVTSYNPSLKGRITISRDTSKNQFSLKLSSV |
| | | TAADTAVYYCARKGTYYRYEGSYWYFDVWGQGTTVTVSS |
| 10 | VH4 variable region | QVQLQESGPGLVKPSQTLSLTCTVSGYSITSDYAWNWIRQHPG |
| | | KGLQWMGYLSYSGVTSYNPSLKGRITISRDTSKNQFFLKLSSV |
| | | TAADTAVYYCARKGTYYRYEGSYWYFDVWGQGTTVTVSS |
| 11 | VL1 variable region | DIQMTQSPSSLSASVGDRVTITCHANQNIDVWLSWYQQKPGKA |
| | | PKLLIYKTSNLHTGVPSRFSGSGSGTDFTFTISSLQPEDIATYYCQ |
| | | QGQSYPLTFGGGTKLEIK |
| 12 | VL2 variable region | DIQMTQSPSSLSASVGDRVTITCHANQNIDVWLSWYQQKPGKA |
| | | PKLLIYKTSNLHTGVPSRFSGSGSGTDFTLTISSLQPEDIATYYCQ |
| | | QGQSYPLTFGGGTKLEIK |
| 13 | VL3 variable region | DIQMTQSPSSLSASVGDRVTITCHANQNIDVWLSWYQQKPGNA |
| | | PKLLIYKTSNLHTGVPSRFSGSGSGTDFTFTISSLQPEDIATYYCQ |
| | | QGQSYPLTFGGGTKLEIK |
| 14 | VL4 variable region | DIQMTQSPSSLSASVGDRVTITCHANQNIDVWLSWYQQKPGNA |
| | | PKLLIYKTSNLHTGVPSRFSGSGSGTDFTLTISSLQPEDIATYYCQ |
| | | QGQSYPLTFGGGTKLEIK |
| 15 | TRPM4 Epitope | RDSDSNCSSEPGFWAHPPGAQ |
| 16 | TRPM4 Epitope | EPGF |
| 17 | Nucleotide sequence | ATGGGCTGGTCATGCATTATTCTGTTTCTGGTCGCAACTGCTAC |
| | encoding VH1 | AGGCGTGCATAGTCAAGTGCAGCTGCAGGAGAGCGGACCTGG |
| | | CCTGGTGAAGCCTTCTCAGACCCTGAGCCTCACCTGTACCGTG |
| | | TCCGGATATAGCATCACAAGCGACTACGCCTGGAATTGGATCA |
| | | GACAGCACCCAGGCAAGGGCCTGGAATGGATCGGCTACCTGTC |
| | | CTACAGCGGCGTGACATCTTATAACCCCAGCCTGAAAGGAAGA |
| | | GTGACCATCAGCGTGGACACCTCTAAGAACCAGTTCAGCCTGA |
| | | AGCTGTCTAGCGTCACAGCCGCTGATACCGCCGTGTACTACTGC |
| | | GCCAGAAAGGGCACCTACTACCGGTACGAGGGCAGCTACTGGT |
| | | ACTTCGACGTGTGGGGCCAGGGCACAACCGTGACCGTTTCCAG |
| | | CGCCAGCACCAAGGGCCCTTCCGTGTTTCCACTGGCCCCCTCCT |
| | | CTAAATCCACATCTGGCGGCACCGCCGCCCTGGGCTGTCTGGTG |
| | | AAGGACTACTTCCCAGAGCCTGTGACAGTGTCCTGGAACTCTGG |
| | | CGCCCTGACATCCGGCGTGCACACATTTCCAGCCGTGCTGCAGA |
| | | GCTCCGGCCTGTACAGCCTGTCTAGCGTGGTGACAGTGCCCTCC |
| | | TCTAGCCTGGGCACACAGACCTATATCTGCAACGTGAATCACAA |
| | | GCCAAGCAATACCAAGGTGGACAAGAAGGTGGAGCCCAAGTCC |
| | | TGTGATAAGACACACACCTGCCCCCCTTGTCCTGCTCCCGAGCT |
| | | GCTGGGCGGCCCTAGCGTGTTCCTGTTTCCACCCAAGCCTAAGG |
| | | ACACCCTGATGATCTCCCGGACACCCGAGGTGACCTGCGTGGT |
| | | GGTGGACGTGTCTCACGAGGATCCTGAGGTGAAGTTCAACTGG |
| | | TATGTGGATGGCGTGGAGGTGCACAATGCCAAGACCAAGCCCA |
| | | GAGAGGAGCAGTACAACTCTACATATAGGGTGGTGAGCGTGCT |
| | | GACCGTGCTGCACCAGGACTGGCTGAACGGCAAGGAGTATAAG |
| | | TGCAAGGTGTCCAATAAGGCCCTGCCCGCCCCCATCGAGAAGA |
| | | CAATCAGCAAGGCCAAGGGCCAGCCTCGGGAGCCACAGGTGTA |
| | | CACCCTGCCTCCATCCAGAGACGAGCTGACAAAGAACCAGGTG |
| | | TCTCTGACATGTCTGGTGAAGGGCTTCTATCCTAGCGATATCGC |
| | | CGTGGAGTGGGAGTCCAATGGCCAGCCAGAGAACAATTACAAG |
| | | ACCACACCCCCTGTGCTGGACTCCGATGGCTCCTTCTTTCTGTAT |
| | | TCCAAGCTGACCGTGGATAAGTCTCGGTGGCAGCAGGGCAACG |
| | | TGTTCAGCTGTTCCGTGATGCACGAAGCCCTGCATAATCACTAT |
| | | ACTCAGAAATCCCTGTCCCTGTCACCTGGAAAGTGATAA |
| 18 | Nucleotide sequence | ATGGGCTGGTCATGCATTATTCTGTTTCTGGTCGCAACTGCTAC |
| | encoding VH2 | AGGCGTGCATAGTCAAGTGCAGCTGCAGGAGAGCGGACCTGG |
| | | CCTGGTGAAGCCATCTCAGACCCTGTCTCTCACATGTACCGTG |
| | | TCTGGATATAGCATCACCAGCGACTACGCCTGGAACTGGATCC |
| | | GGCAGCACCCCGGCAAGGGCCTGGAATGGATGGGCTACCTGT |
| | | CCTACAGCGGAGTTACATCTTATAATCCTAGCCTGAAAGGCAG |
| | | AGTGACCATCAGCAGAGATACCTCCAAGAACCAGTTCAGCCT |
| | | GAAGCTGAGCAGCGTGACAGCCGCTGACACCGCCGTGTACTA |
| | | CTGCGCCAGAAAGGGCACATACTACCGGTACGAGGGCAGCTA |
| | | CTGGTACTTCGACGTGTGGGGCCAGGGCACCACCGTCACAGT |
| | | GTCCAGCGCCAGCACCAAGGGCCCTTCCGTGTTTCCACTGGC |
| | | CCCCTCCTCTAAATCCACATCTGGCGGCACCGCCGCCCTGGG |
| | | CTGTCTGGTGAAGGACTACTTCCCAGAGCCTGTGACAGTGTC |
| | | CTGGAACTCTGGCGCCCTGACATCCGGCGTGCACACATTTCC |
| | | AGCCGTGCTGCAGAGCTCCGGCCTGTACAGCCTGTCTAGCGT |
| | | GGTGACAGTGCCCTCCTCTAGCCTGGGCACACAGACCTATAT |
| | | CTGCAACGTGAATCACAAGCCAAGCAATACCAAGGTGGACA |
| | | AGAAGGTGGAGCCCAAGTCCTGTGATAAGACACACACCTGC |
| | | CCCCCTTGTCCTGCTCCCGAGCTGCTGGGCGGCCCTAGCGTG |
| | | TTCCTGTTTCCACCCAAGCCTAAGGACACCCTGATGATCTCC |
| | | CGGACACCCGAGGTGACCTGCGTGGTGGTGGACGTGTCTCA |
| | | CGAGGATCCTGAGGTGAAGTTCAACTGGTATGTGGATGGCG |
| | | TGGAGGTGCACAATGCCAAGACCAAGCCCAGAGAGGAGCA |
| | | GTACAACTCTACATATAGGGTGGTGAGCGTGCTGACCGTGC |
| | | TGCACCAGGACTGGCTGAACGGCAAGGAGTATAAGTGCAA |
| | | GGTGTCCAATAAGGCCCTGCCCGCCCCCATCGAGAAGACA |
| | | ATCAGCAAGGCCAAGGGCCAGCCTCGGGAGCCACAGGTGT |
| | | ACACCCTGCCTCCATCCAGAGACGAGCTGACAAAGAACCA |
| | | GGTGTCTCTGACATGTCTGGTGAAGGGCTTCTATCCTAGCG |
| | | ATATCGCCGTGGAGTGGGAGTCCAATGGCCAGCCAGAGAA |
| | | CAATTACAAGACCACACCCCCTGTGCTGGACTCCGATGGC |
| | | TCCTTCTTTCTGTATTCCAAGCTGACCGTGGATAAGTCTCG |
| | | GTGGCAGCAGGGCAACGTGTTCAGCTGTTCCGTGATGCAC |
| | | GAAGCCCTGCATAATCACTATACTCAGAAATCCCTGTCCCT |
| | | GTCACCTGGAAAGTGATAA |
| 19 | Nucleotide sequence | ATGGGCTGGTCATGCATTATTCTGTTTCTGGTCGCAACTGC |
| | encoding VH3 | TACAGGCGTGCATAGTCAAGTGCAGCTGCAGGAGTCTGGA |
| | | CCTGGACTGGTGAAGCCTAGCCAGACCCTGAGCCTGACCT |
| | | GTACCGTCTCCGGCTACAGCATCACCTCCGACTACGCCTGG |
| | | AATTGGATCAGACAGCACCCCGGCAAGGGCCTCCAGTGGA |
| | | TGGGCTACCTGTCTTATTCTGGAGTGACATCTTACAACCCA |
| | | AGCCTGAAAGGCAGAATCACCATCAGCCGGGACACCAGCA |
| | | AGAACCAGTTCAGCCTGAAGCTGAGCAGCGTGACCGCCGC |
| | | TGATACAGCCGTGTACTACTGCGCCAGAAAGGGCACCTAC |
| | | TACCGGTACGAAGGCAGCTATTGGTACTTCGACGTGTGGG |
| | | GCCAGGGCACAACAGTGACCGTGTCCAGCGCCAGCACCAA |
| | | GGGCCCTTCCGTGTTTCCACTGGCCCCCTCCTCTAAATCCA |
| | | CATCTGGCGGCACCGCCGCCCTGGGCTGTCTGGTGAAGGA |
| | | CTACTTCCCAGAGCCTGTGACAGTGTCCTGGAACTCTGGCG |
| | | CCCTGACATCCGGCGTGCACACATTTCCAGCCGTGCTGCAG |
| | | AGCTCCGGCCTGTACAGCCTGTCTAGCGTGGTGACAGTGCC |
| | | CTCCTCTAGCCTGGGCACACAGACCTATATCTGCAACGTGA |
| | | ATCACAAGCCAAGCAATACCAAGGTGGACAAGAAGGTGGA |
| | | GCCCAAGTCCTGTGATAAGACACACACCTGCCCCCCTTGTC |
| | | CTGCTCCCGAGCTGCTGGGCGGCCCTAGCGTGTTCCTGTTT |
| | | CCACCCAAGCCTAAGGACACCCTGATGATCTCCCGGACAC |
| | | CCGAGGTGACCTGCGTGGTGGTGGACGTGTCTCACGAGGA |
| | | TCCTGAGGTGAAGTTCAACTGGTATGTGGATGGCGTGGAG |
| | | GTGCACAATGCCAAGACCAAGCCCAGAGAGGAGCAGTAC |
| | | AACTCTACATATAGGGTGGTGAGCGTGCTGACCGTGCTGC |
| | | ACCAGGACTGGCTGAACGGCAAGGAGTATAAGTGCAAGG |
| | | TGTCCAATAAGGCCCTGCCCGCCCCCATCGAGAAGACAAT |
| | | CAGCAAGGCCAAGGGCCAGCCTCGGGAGCCACAGGTGTA |
| | | CACCCTGCCTCCATCCAGAGACGAGCTGACAAAGAACCA |
| | | GGTGTCTCTGACATGTCTGGTGAAGGGCTTCTATCCTAGC |
| | | GATATCGCCGTGGAGTGGGAGTCCAATGGCCAGCCAGAG |
| | | AACAATTACAAGACCACACCCCCTGTGCTGGACTCCGATG |
| | | GCTCCTTCTTTCTGTATTCCAAGCTGACCGTGGATAAGTCT |
| | | CGGTGGCAGCAGGGCAACGTGTTCAGCTGTTCCGTGATGC |
| | | ACGAAGCCCTGCATAATCACTATACTCAGAAATCCCTGTC |
| | | CCTGTCACCTGGAAAGTGATAA |
| 20 | Nucleotide sequence | ATGGGCTGGTCATGCATTATTCTGTTTCTGGTCGCAACTGCT |
| | encoding VH4 | ACAGGCGTGCATAGTCAAGTGCAGCTGCAGGAGAGCGGACC |
| | | TGGCCTGGTGAAGCCTAGCCAGACCCTGTCCCTGACATGTAC |
| | | CGTGTCTGGCTACAGCATCACAAGCGACTACGCCTGGAATTG |
| | | GATCCGGCAGCACCCAGGAAAGGGCCTCCAGTGGATGGGCT |
| | | ATCTGAGCTATAGCGGCGTGACATCCTACAACCCCAGCCTGA |
| | | AAGGAAGAATCACCATCAGCAGAGATACATCTAAGAACCAG |
| | | TTTTTCCTGAAGCTGTCTAGCGTGACCGCCGCTGACACCGCC |
| | | GTGTACTACTGCGCCAGAAAGGGCACCTACTACCGGTACGAA |
| | | GGCAGCTACTGGTACTTCGACGTGTGGGGCCAGGGCACAACC |
| | | GTCACCGTGTCCAGCGCCAGCACCAAGGGCCCTTCCGTGTTTC |
| | | CACTGGCCCCCTCCTCTAAATCCACATCTGGCGGCACCGCCGC |
| | | CCTGGGCTGTCTGGTGAAGGACTACTTCCCAGAGCCTGTGACA |
| | | GTGTCCTGGAACTCTGGCGCCCTGACATCCGGCGTGCACACAT |
| | | TTCCAGCCGTGCTGCAGAGCTCCGGCCTGTACAGCCTGTCTAG |
| | | CGTGGTGACAGTGCCCTCCTCTAGCCTGGGCACACAGACCTAT |
| | | ATCTGCAACGTGAATCACAAGCCAAGCAATACCAAGGTGGAC |
| | | AAGAAGGTGGAGCCCAAGTCCTGTGATAAGACACACACCTGC |
| | | CCCCCTTGTCCTGCTCCCGAGCTGCTGGGCGGCCCTAGCGTGT |
| | | TCCTGTTTCCACCCAAGCCTAAGGACACCCTGATGATCTCCCG |
| | | GACACCCGAGGTGACCTGCGTGGTGGTGGACGTGTCTCACGA |
| | | GGATCCTGAGGTGAAGTTCAACTGGTATGTGGATGGCGTGGA |
| | | GGTGCACAATGCCAAGACCAAGCCCAGAGAGGAGCAGTACA |
| | | ACTCTACATATAGGGTGGTGAGCGTGCTGACCGTGCTGCACC |
| | | AGGACTGGCTGAACGGCAAGGAGTATAAGTGCAAGGTGTCC |
| | | AATAAGGCCCTGCCCGCCCCCATCGAGAAGACAATCAGCAAG |
| | | GCCAAGGGCCAGCCTCGGGAGCCACAGGTGTACACCCTGCCT |
| | | CCATCCAGAGACGAGCTGACAAAGAACCAGGTGTCTCTGACA |
| | | TGTCTGGTGAAGGGCTTCTATCCTAGCGATATCGCCGTGGAGT |
| | | GGGAGTCCAATGGCCAGCCAGAGAACAATTACAAGACCACAC |
| | | CCCCTGTGCTGGACTCCGATGGCTCCTTCTTTCTGTATTCCAAG |
| | | CTGACCGTGGATAAGTCTCGGTGGCAGCAGGGCAACGTGTTCA |
| | | GCTGTTCCGTGATGCACGAAGCCCTGCATAATCACTATACTCA |
| | | GAAATCCCTGTCCCTGTCACCTGGAAAGTGATAA |
| 21 | Nucleotide sequence | ATGGGCTGGTCATGTATTATTCTGTTTCTGGTCGCAACTGCTAC |
| | encoding VL1 | AGGGGTCCATAGTGATATTCAGATGACCCAGAGCCCCAGCAGC |
| | | CTCTCCGCCAGCGTGGGAGATAGAGTGACCATCACCTGTCACG |
| | | CCAACCAGAATATCGACGTCTGGCTGAGCTGGTATCAGCAGAA |
| | | ACCTGGCAAGGCCCCTAAGCTGCTGATCTACAAGACCAGCAAC |
| | | CTGCACACCGGCGTGCCATCTCGGTTCAGCGGCTCTGGCAGCG |
| | | GCACCGACTTCACATTTACAATCAGCTCTCTGCAGCCTGAGGA |
| | | CATCGCTACATACTACTGCCAGCAAGGCCAGTCCTACCCCCTG |
| | | ACCTTCGGCGGAGGCACAAAGCTGGAAATCAAGAGGACAGTG |
| | | GCCGCCCCAAGCGTGTTCATCTTTCCCCCTTCCGACGAGCAGCT |
| | | GAAGTCTGGCACCGCCAGCGTGGTGTGCCTGCTGAACAACTTCT |
| | | ACCCTCGGGAGGCCAAGGTCCAGTGGAAGGTGGATAACGCCCT |
| | | GCAGTCTGGCAATAGCCAGGAGTCCGTGACCGAGCAGGACTCT |
| | | AAGGATAGCACATATTCCCTGTCTAGCACCCTGACACTGAGCA |
| | | AGGCCGATTACGAGAAGCACAAGGTGTATGCCTGTGAAGTCAC |
| | | CCATCAGGGGCTGTCATCACCCGTCACTAAGTCATTCAATCGCG |
| | | GAGAATGCTGATAA |
| 22 | Nucleotide sequence | ATGGGCTGGTCATGTATTATTCTGTTTCTGGTCGCAACTGCTAC |
| | encoding VL2 | AGGGGTCCATAGTGATATTCAGATGACCCAGTCCCCAAGCAGC |
| | | CTCTCCGCCAGCGTGGGCGATAGAGTGACCATCACCTGTCACG |
| | | CCAACCAGAATATCGACGTGTGGCTGAGCTGGTACCAGCAAAA |
| | | GCCCGGCAAAGCCCCTAAGCTGCTGATCTACAAGACCAGCAAC |
| | | CTGCACACCGGCGTCCCCAGCCGGTTCAGCGGATCTGGCAGCG |
| | | GCACCGACTTCACCCTGACAATCAGCTCTCTGCAGCCTGAGGA |
| | | CATCGCTACATACTACTGCCAGCAGGGCCAGTCTTATCCTCTGA |
| | | CATTTGGCGGCGGAACAAAGCTGGAAATCAAGAGGACAGTGGC |
| | | CGCCCCAAGCGTGTTCATCTTTCCCCCTTCCGACGAGCAGCTGA |
| | | AGTCTGGCACCGCCAGCGTGGTGTGCCTGCTGAACAACTTCTAC |
| | | CCTCGGGAGGCCAAGGTCCAGTGGAAGGTGGATAACGCCCTGC |
| | | AGTCTGGCAATAGCCAGGAGTCCGTGACCGAGCAGGACTCTAA |
| | | GGATAGCACATATTCCCTGTCTAGCACCCTGACACTGAGCAAG |
| | | GCCGATTACGAGAAGCACAAGGTGTATGCCTGTGAAGTCACCC |
| | | ATCAGGGGCTGTCATCACCCGTCACTAAGTCATTCAATCGCGG |
| | | AGAATGCTGATAA |
| 23 | Nucleotide sequence | ATGGGCTGGTCATGTATTATTCTGTTTCTGGTCGCAACTGCTAC |
| | encoding VL3 | AGGGGTCCATAGTGATATTCAGATGACCCAGAGCCCCAGCAG |
| | | CCTGTCCGCCAGCGTCGGCGATAGAGTGACAATCACCTGTCA |
| | | CGCCAACCAGAATATCGACGTGTGGCTGTCTTGGTATCAGCA |
| | | AAAACCTGGCAACGCTCCTAAGCTCCTGATCTACAAGACCAG |
| | | CAACCTGCACACAGGCGTGCCAAGCCGGTTCAGCGGCAGCGG |
| | | ATCTGGCACCGACTTCACCTTCACAATCAGCTCTCTGCAGCCTG |
| | | AGGACATCGCCACCTACTACTGCCAGCAGGGCCAGTCCTACCC |
| | | CCTGACCTTTGGCGGAGGCACAAAGCTGGAAATCAAGAGGACA |
| | | GTGGCCGCCCCAAGCGTGTTCATCTTTCCCCCTTCCGACGAGCA |
| | | GCTGAAGTCTGGCACCGCCAGCGTGGTGTGCCTGCTGAACAACT |
| | | TCTACCCTCGGGAGGCCAAGGTCCAGTGGAAGGTGGATAACGC |
| | | CCTGCAGTCTGGCAATAGCCAGGAGTCCGTGACCGAGCAGGAC |
| | | TCTAAGGATAGCACATATTCCCTGTCTAGCACCCTGACACTGAG |
| | | CAAGGCCGATTACGAGAAGCACAAGGTGTATGCCTGTGAAGTC |
| | | ACCCATCAGGGGCTGTCATCACCCGTCACTAAGTCATTCAATCG |
| | | CGGAGAATGCTGATAA |
| 24 | Nucleotide sequence | ATGGGCTGGTCATGTATTATTCTGTTTCTGGTCGCAACTGCTA |
| | encoding VL4 | CAGGGGTCCATAGTGATATTCAGATGACCCAGTCTCCATCTA |
| | | GCCTCTCCGCCAGCGTGGGCGACCGGGTGACCATCACCTGTCA |
| | | CGCCAACCAGAACATCGACGTGTGGCTGAGCTGGTATCAGCA |
| | | GAAACCTGGAAATGCCCCTAAGCTGCTGATCTACAAGACCAG |
| | | CAACCTGCACACCGGCGTCCCCAGCAGATTCAGCGGCAGCGG |
| | | CTCTGGCACCGACTTCACCCTGACAATCAGCAGCCTGCAGCCT |
| | | GAGGATATCGCTACATACTACTGCCAGCAAGGCCAGTCCTACC |
| | | CCCTGACATTTGGCGGCGGAACAAAGCTGGAAATCAAGAGGA |
| | | CAGTGGCCGCCCCAAGCGTGTTCATCTTTCCCCCTTCCGACGA |
| | | GCAGCTGAAGTCTGGCACCGCCAGCGTGGTGTGCCTGCTGAA |
| | | CAACTTCTACCCTCGGGAGGCCAAGGTCCAGTGGAAGGTGGA |
| | | TAACGCCCTGCAGTCTGGCAATAGCCAGGAGTCCGTGACCGA |
| | | GCAGGACTCTAAGGATAGCACATATTCCCTGTCTAGCACCCT |
| | | GACACTGAGCAAGGCCGATTACGAGAAGCACAAGGTGTATG |
| | | CCTGTGAAGTCACCCATCAGGGGCTGTCATCACCCGTCACTA |
| | | AGTCATTCAATCGCGGAGAATGCTGATAA |
| 25 | VH | QVQLQESGPGLVKPSQSLSLTCTVTGYSITSDYAWNWIRQFPGN |
| | | KLQWMGYLSYSGVTSYNPSLKGRISITRDTSKNQFFLQLSSVTP |
| | | EDTATYYCARKGTYYRYEGSYWYFDVWGAGTTVTVSS |
| 26 | VL | DIQMNQSPSSLSASLGDTITITCHANQNIDVWLSWYQQKPGNV |
| | | PKLLIYKTSNLHTGVPSRFSGSGSGTDFTLTISSLQPEDIATYYC |
| | | QQGQSYPLTFGGGTKLEIK |
| 27 | Nucleotide sequence | ATGGGCTGGTCATGCATTATTCTGTTTCTGGTCGCAACTGC |
| | encoding VH | TACAGGCGTGCATAGTCAGGTGCAGCTGCAGGAGAGCGGA |
| | | CCTGGCCTGGTGAAGCCTAGCCAGAGCCTGTCCCTCACCTG |
| | | TACCGTGACCGGCTACTCCATCACAAGCGACTACGCCTGGA |
| | | ATTGGATCAGACAGTTTCCAGGCAACAAGCTGCAGTGGATG |
| | | GGCTACCTGTCTTATTCTGGCGTGACATCTTACAACCCCAGC |
| | | CTGAAAGGCAGAATCAGCATCACCCGGGACACCAGCAAGAA |
| | | CCAGTTCTTCCTGCAACTGAGCAGCGTGACCCCTGAAGATA |
| | | CCGCCACATACTACTGCGCCAGAAAGGGCACCTACTACCGG |
| | | TACGAGGGCAGCTATTGGTACTTCGACGTGTGGGGCGCTGG |
| | | AACAACCGTCACAGTGTCCAGCGCCAGCACCAAGGGCCCTT |
| | | CCGTGTTTCCACTGGCCCCCTCCTCTAAATCCACATCTGGCG |
| | | GCACCGCCGCCCTGGGCTGTCTGGTGAAGGACTACTTCCCA |
| | | GAGCCTGTGACAGTGTCCTGGAACTCTGGCGCCCTGACATCC |
| | | GGCGTGCACACATTTCCAGCCGTGCTGCAGAGCTCCGGCCT |
| | | GTACAGCCTGTCTAGCGTGGTGACAGTGCCCTCCTCTAGCC |
| | | TGGGCACACAGACCTATATCTGCAACGTGAATCACAAGCC |
| | | AAGCAATACCAAGGTGGACAAGAAGGTGGAGCCCAAGTC |
| | | CTGTGATAAGACACACACCTGCCCCCCTTGTCCTGCTCCCG |
| | | AGCTGCTGGGCGGCCCTAGCGTGTTCCTGTTTCCACCCAAG |
| | | CCTAAGGACACCCTGATGATCTCCCGGACACCCGAGGTGA |
| | | CCTGCGTGGTGGTGGACGTGTCTCACGAGGATCCTGAGGT |
| | | GAAGTTCAACTGGTATGTGGATGGCGTGGAGGTGCACAAT |
| | | GCCAAGACCAAGCCCAGAGAGGAGCAGTACAACTCTACA |
| | | TATAGGGTGGTGAGCGTGCTGACCGTGCTGCACCAGGACT |
| | | GGCTGAACGGCAAGGAGTATAAGTGCAAGGTGTCCAATA |
| | | AGGCCCTGCCCGCCCCCATCGAGAAGACAATCAGCAAGG |
| | | CCAAGGGCCAGCCTCGGGAGCCACAGGTGTACACCCTGC |
| | | CTCCATCCAGAGACGAGCTGACAAAGAACCAGGTGTCTCT |
| | | GACATGTCTGGTGAAGGGCTTCTATCCTAGCGATATCGCC |
| | | GTGGAGTGGGAGTCCAATGGCCAGCCAGAGAACAATTAC |
| | | AAGACCACACCCCCTGTGCTGGACTCCGATGGCTCCTTCT |
| | | TTCTGTATTCCAAGCTGACCGTGGATAAGTCTCGGTGGCA |
| | | GCAGGGCAACGTGTTCAGCTGTTCCGTGATGCACGAAGC |
| | | CCTGCATAATCACTATACTCAGAAATCCCTGTCCCTGTCA |
| | | CCTGGAAAGTGATAA |
| 28 | Nucleotide sequence | ATGGGCTGGTCATGTATTATTCTGTTTCTGGTCGCAACTG |
| | encoding VL | CTACAGGGGTCCATAGTGATATCCAGATGAACCAGAGCC |
| | | CTTCTAGCCTCTCCGCCAGCCTGGGCGACACCATTACAAT |
| | | CACCTGTCACGCTAATCAGAACATCGACGTGTGGCTGAG |
| | | CTGGTATCAGCAGAAACCTGGCAACGTGCCCAAGCTGCT |
| | | GATCTACAAGACCAGCAACCTGCACACCGGCGTGCCATC |
| | | TAGATTCAGCGGATCTGGCAGCGGCACCGACTTCACCCTG |
| | | ACCATCAGCAGCCTGCAGCCTGAGGATATCGCCACATAC |
| | | TACTGCCAGCAAGGCCAGTCCTACCCCCTGACATTTGGCG |
| | | GCGGAACAAAGCTGGAAATCAAGAGGACAGTGGCCGCCC |
| | | CAAGCGTGTTCATCTTTCCCCCTTCCGACGAGCAGCTGAAG |
| | | TCTGGCACCGCCAGCGTGGTGTGCCTGCTGAACAACTTCT |
| | | ACCCTCGGGAGGCCAAGGTCCAGTGGAAGGTGGATAACG |
| | | CCCTGCAGTCTGGCAATAGCCAGGAGTCCGTGACCGAGC |
| | | AGGACTCTAAGGATAGCACATATTCCCTGTCTAGCACCC |
| | | TGACACTGAGCAAGGCCGATTACGAGAAGCACAAGGTG |
| | | TATGCCTGTGAAGTCACCCATCAGGGGCTGTCATCACCC |
| | | GTCACTAAGTCATTCAATCGCGGAGAATGCTGATAA |
| 29 | VH FR1 | QVQLQESGPGLVKPSQSLSLTCTVT |
| 30 | VH FR2 | WIRQFPGNKLQWMG |
| 31 | VH FR3 | RISITRDTSKNQFFLQLSSVTPEDTATYYCAR |
| 32 | VH FR4 | WGAGTTVTVSS |
| 33 | VH1/VH2/VH3/VH4 FR1 | QVQLQESGPGLVKPSQTLSLTCTVS |
| 34 | VH1 FR2 | WIRQHPGKGLEWIG |
| 35 | VH1 FR3 | RVTISVDTSKNQFSLKLSSVTAADTAVYYCAR |
| 36 | VH1/VH2/VH3/VH4 FR4 | WGQGTTVTVSS |
| 37 | VH2 FR2 | WIRQHPGKGLEWMG |
| 38 | VH2 FR3 | RVTISRDTSKNQFSLKLSSVTAADTAVYYCAR |
| 39 | VH3/VH4 FR2 | WIRQHPGKGLQWMG |
| 40 | VH3 FR3 | RITISRDTSKNQFSLKLSSVTAADTAVYYCAR |
| 41 | VH4 FR3 | RITISRDTSKNQFFLKLSSVTAADTAVYYCAR |
| 42 | VL FR1 | DIQMNQSPSSLSASLGDTITITC |
| 43 | VL FR2 | WYQQKPGNVPKLLIY |
| 44 | VL/VL2/VL4 FR3 | GVPSRFSGSGSGTDFTLTISSLQPEDIATYYC |
| 45 | Light chain FR4 | FGGGTKLEIK |
| 46 | VL1/VL2/VL3/VL4 FR1 | DIQMTQSPSSLSASVGDRVTITC |
| 47 | VL1/VL2 FR2 | WYQQKPGKAPKLLIY |
| 48 | VL1/VL3 FR3 | GVPSRFSGSGSGTDFTFTISSLQPEDIATYYC |
| 49 | VL3/VL4 FR2 | WYQQKPGNAPKLLIY |
| 50 | VH1 | MGWSCIILFLVATATGVHSQVQLQESGPGLVKPSQTLSLT |
| | | CTVSGYSITSDYAWNWIRQHPGKGLEWIGYLSYSGVTSY |
| | | NPSLKGRVTISVDTSKNQFSLKLSSVTAADTAVYYCARK |
| | | GTYYRYEGSYWYFDVWGQGTTVTVSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSL |
| | | SPGK |
| 51 | VH2 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCAR |
| | | KGTYYRYEGSYWYFDVWGQGTTVTVSSASTKGPSVFPL |
| | | APSSKSTSGGTAALGCLVKDYFPEPVTVSWNSGALTSGV |
| | | HTFPAVLQSSGLYSLSSVVTVPSSSLGTQTYICNVNHKPS |
| | | NTKVDKKVEPKSCDKTHTCPPCPAPELLGGPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPSRDELTKNQ |
| | | VSLTCLVKGFYPSDIAVEWESNGQPENNYKTTPPVLDSDG |
| | | SFFLYSKLTVDKSRWQQGNVFSCSVMHEALHNHYTQKS |
| | | LSLSPGK |
| 52 | VH3 | MGWSCIILFLVATATGVHSQVQLQESGPGLVKPSQTLSLT |
| | | CTVSGYSITSDYAWNWIRQHPGKGLQWMGYLSYSGVTS |
| | | YNPSLKGRITISRDTSKNQFSLKLSSVTAADTAVYYCARK |
| | | GTYYRYEGSYWYFDVWGQGTTVTVSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSL |
| | | SPGK |
| 53 | VH4 | MGWSCIILFLVATATGVHSQVQLQESGPGLVKPSQTLSLT |
| | | CTVSGYSITSDYAWNWIRQHPGKGLQWMGYLSYSGVTS |
| | | YNPSLKGRITISRDTSKNQFFLKLSSVTAADTAVYYCARK |
| | | GTYYRYEGSYWYFDVWGQGTTVTVSSASTKGPSVFPLAP |
| | | SSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTK |
| | | PREEQYNSTYRVVSVLTVLHQDWLNGKEYKCKVSNKALP |
| | | APIEKTISKAKGQPREPQVYTLPPSRDELTKNQVSLTCLVK |
| | | GFYPSDIAVEWESNGQPENNYKTTPPVLDSDGSFFLYSKLT |
| | | VDKSRWQQGNVFSCSVMHEALHNHYTQKSLSLSPGK |
| 54 | VH5 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCARK |
| | | GTYYRYEGSYWYFDVWGQGTTVTVSSASTKGPSVFPLAP |
| | | SSKSTSGGTAALGCLVKDYFPEPVTVSWNSGALTSGVHTF |
| | | PAVLQSSGLYSLSSVVTVPSSSLGTQTYICNVNHKPSNTKV |
| | | DKKVEPKSCDKTHTCPPCPAPEAAGGPSVFLFPPKPKDTL |
| | | MISRTPEVTCVVVDVSHEDPEVKFNWYVDGVEVHNAKT |
| | | KPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLY |
| | | SKLTVDKSRWQQGNVFSCSVMHEALHNHYTQKSLSLSPGK |
| 55 | VH6 | MGWSCIILFLVATATGVHSQVQLQESGPGLVKPSQTLSLT |
| | | CTVSGYSITSDYAWNWIRQHPGKGLEWMGYLSYSGVTSY |
| | | NPSLKGRVTISRDTSKNQFSLKLSSVTAADTAVYYCARKG |
| | | TYYRYEGSYWYFDVWGQGTTVTVSSASTKGPSVFPLAPS |
| | | SKSTSGGTAALGCLVKDYFPEPVTVSWNSGALTSGVHTFP |
| | | AVLQSSGLYSLSSVVTVPSSSLGTQTYICNVNHKPSNTKVD |
| | | KKVEPKSCDKTHTCPPCPAPELLGGPSVFLFPPKPKDTLMI |
| | | SRTPEVTCVVVDVSHEDPEVKFNWYVDGVEVHNAKTKPR |
| | | EEQYASTYRVVSVLTVLHQDWLNGKEYKCKVSNKALPAPI |
| | | EKTISKAKGQPREPQVYTLPPSRDELTKNQVSLTCLVKGFY |
| | | PSDIAVEWESNGQPENNYKTTPPVLDSDGSFFLYSKLTVDK |
| | | SRWQQGNVFSCSVMHEALHNHYTQKSLSLSPGK |
| 56 | VH7 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCARK |
| | | GTYYRYEGSYWYFDVWGQGTTVTVSSASTKGPSVFPLAP |
| | | CSRSTSESTAALGCLVKDYFPEPVTVSWNSGALTSGVHTF |
| | | PAVLQSSGLYSLSSVVTVPSSSLGTKTYTCNVDHKPSNTK |
| | | VDKRVESKYGPPCPPCPAPEFLGGPSVFLFPPKPKDTLMIS |
| | | RTPEVTCVVVDVSQEDPEVQFNWYVDGVEVHNAKTKPR |
| | | EEQFNSTYRVVSVLTVLHQDWLNGKEYKCKVSNKGLPSS |
| | | IEKTISKAKGQPREPQVYTLPPSQEEMTKNQVSLTCLVKG |
| | | FYPSDIAVEWESNGQPENNYKTTPPVLDSDGSFFLYSRLT |
| | | VDKSRWQEGNVFSCSVMHEALHNHYTQKSLSLSLGK |
| 57 | VL1 | MGWSCIILFLVATATGVHSDIQMTQSPSSLSASVGDRVTI |
| | | TCHANQNIDVWLSWYQQKPGKAPKLLIYKTSNLHTGVP |
| | | SRFSGSGSGTDFTFTISSLQPEDIATYYCQQGQSYPLTFGG |
| | | GTKLEIKRTVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 58 | VL2 | MGWSCIILFLVATATGVHSDIQMTQSPSSLSASVGDRVTI |
| | | TCHANQNIDVWLSWYQQKPGKAPKLLIYKTSNLHTGVP |
| | | SRFSGSGSGTDFTLTISSLQPEDIATYYCQQGQSYPLTFGG |
| | | GTKLEIKRTVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 59 | VL3 | MGWSCIILFLVATATGVHSDIQMTQSPSSLSASVGDRVTI |
| | | TCHANQNIDVWLSWYQQKPGNAPKLLIYKTSNLHTGVP |
| | | SRFSGSGSGTDFTFTISSLQPEDIATYYCQQGQSYPLTFGG |
| | | GTKLEIKRTVAAPSVFIFPPSDEQLKSGTASVVCLLNNFY |
| | | PREAKVQWKVDNALQSGNSQESVTEQDSKDSTYSLSST |
| | | LTLSKADYEKHKVYACEVTHQGLSSPVTKSFNRGEC |
| 60 | VL4 | MGWSCIILFLVATATGVHSDIQMTQSPSSLSASVGDRVTI |
| | | TCHANQNIDVWLSWYQQKPGNAPKLLIYKTSNLHTGVP |
| | | SRFSGSGSGTDFTLTISSLQPEDIATYYCQQGQSYPLTFG |
| | | GGTKLEIKRTVAAPSVFIFPPSDEQLKSGTASVVCLLNNF |
| | | YPREAKVQWKVDNALQSGNSQESVTEQDSKDSTYSLSS |
| | | TLTLSKADYEKHKVYACEVTHQGLSSPVTKSFNRGEC |
| 61 | Nucleotide sequence | ATGGGCTGGTCATGCATTATTCTGTTTCTGGTCGCAAC |
| | encoding VH5 | TGCTACAGGCGTGCATAGTCAAGTGCAGCTGCAGGAG |
| | | AGCGGACCTGGCCTGGTGAAGCCATCTCAGACCCTGT |
| | | CTCTCACATGTACCGTGTCTGGATATAGCATCACCAGC |
| | | GACTACGCCTGGAACTGGATCCGGCAGCACCCCGGCA |
| | | AGGGCCTGGAATGGATGGGCTACCTGTCCTACAGCGG |
| | | AGTTACATCTTATAATCCTAGCCTGAAAGGCAGAGTGA |
| | | CCATCAGCAGAGATACCTCCAAGAACCAGTTCAGCCT |
| | | GAAGCTGAGCAGCGTGACAGCCGCTGACACCGCCGTG |
| | | TACTACTGCGCCAGAAAGGGCACATACTACCGGTACGA |
| | | GGGCAGCTACTGGTACTTCGACGTGTGGGGCCAGGGCA |
| | | CCACCGTCACAGTGTCCAGCGCCAGCACAAAGGGCCC |
| | | TAGCGTGTTTCCACTGGCCCCCTCCTCTAAGTCCACCT |
| | | CTGGAGGAACAGCCGCCCTGGGCTGTCTGGTGAAGGAT |
| | | TATTTCCCAGAGCCCGTGACCGTGTCCTGGAACTCTGGC |
| | | GCCCTGACCAGCGGAGTGCACACATTTCCAGCCGTGCTG |
| | | CAGAGCTCCGGACTGTACTCCCTGTCTAGCGTGGTGACC |
| | | GTGCCTTCCTCTAGCCTGGGCACCCAGACATATATCTGC |
| | | AACGTGAATCACAAGCCCTCCAATACAAAGGTGGACAA |
| | | GAAGGTGGAGCCTAAGTCTTGTGATAAGACCCACACATG |
| | | CCCCCCTTGTCCTGCACCAGAGGCAGCAGGAGGACCTT |
| | | CCGTGTTCCTGTTTCCACCCAAGCCAAAGGACACCCTG |
| | | ATGATCAGCCGCACCCCTGAGGTGACATGCGTGGTGGT |
| | | GGACGTGTCCCACGAGGATCCAGAGGTGAAGTTTAACT |
| | | GGTACGTGGATGGCGTGGAGGTGCACAATGCCAAGACC |
| | | AAGCCTCGGGAGGAGCAGTACAACTCTACCTATAGAGTG |
| | | GTGAGCGTGCTGACAGTGCTGCACCAGGACTGGCTGAAC |
| | | GGCAAGGAGTATAAGTGCAAGGTGTCTAATAAGGCCCT |
| | | GCCCGCCCCTATCGAGAAGACCATCAGCAAGGCAAAGG |
| | | GACAGCCTAGGGAGCCACAGGTGTACACACTGCCTCCA |
| | | TCTAGAGACGAGCTGACCAAGAACCAGGTGAGCCTGACA |
| | | TGTCTGGTGAAGGGCTTCTATCCAAGCGATATCGCCGTG |
| | | GAGTGGGAGTCCAATGGCCAGCCCGAGAACAATTACAAG |
| | | ACCACACCCCCTGTGCTGGACAGCGATGGCTCCTTCTTTC |
| | | TGTATTCCAAGCTGACCGTGGATAAGTCTCGGTGGCAGC |
| | | AGGGCAACGTGTTTAGCTGTTCCGTGATGCACGAGGCCC |
| | | TGCACAATCACTACACCCAGAAGTCTCTGAGCCTGTCC |
| | | CCCGGCAAGTGA |
| 62 | Nucleotide sequence | ATGGGCTGGTCATGCATTATTCTGTTTCTGGTCGCAAC |
| | encoding VH6 | TGCTACAGGCGTGCATAGTCAAGTGCAGCTGCAGGAGA |
| | | GCGGACCTGGCCTGGTGAAGCCATCTCAGACCCTGTCT |
| | | CTCACATGTACCGTGTCTGGATATAGCATCACCAGCGAC |
| | | TACGCCTGGAACTGGATCCGGCAGCACCCCGGCAAGGG |
| | | CCTGGAATGGATGGGCTACCTGTCCTACAGCGGAGTTA |
| | | CATCTTATAATCCTAGCCTGAAAGGCAGAGTGACCATC |
| | | AGCAGAGATACCTCCAAGAACCAGTTCAGCCTGAAGCT |
| | | GAGCAGCGTGACAGCCGCTGACACCGCCGTGTACTACT |
| | | GCGCCAGAAAGGGCACATACTACCGGTACGAGGGCAG |
| | | CTACTGGTACTTCGACGTGTGGGGCCAGGGCACCACCG |
| | | TCACAGTGTCCAGCGCCAGCACCAAGGGCCCTTCCGTG |
| | | TTTCCACTGGCCCCCTCCTCTAAATCCACATCTGGCGGC |
| | | ACCGCCGCCCTGGGCTGTCTGGTGAAGGACTACTTCCC |
| | | AGAGCCTGTGACAGTGTCCTGGAACTCTGGCGCCCTGA |
| | | CATCCGGCGTGCACACATTTCCAGCCGTGCTGCAGAGCT |
| | | CCGGCCTGTACAGCCTGTCTAGCGTGGTGACAGTGCCC |
| | | TCCTCTAGCCTGGGCACACAGACCTATATCTGCAACGT |
| | | GAATCACAAGCCAAGCAATACCAAGGTGGACAAGAAG |
| | | GTGGAGCCCAAGTCCTGTGATAAGACACACACCTGCCC |
| | | CCCTTGTCCTGCTCCCGAGCTGCTGGGCGGCCCTAGCG |
| | | TGTTCCTGTTTCCACCCAAGCCTAAGGACACCCTGATG |
| | | ATCTCCCGGACACCCGAGGTGACCTGCGTGGTGGTGGA |
| | | CGTGTCTCACGAGGATCCTGAGGTGAAGTTCAACTGGT |
| | | ATGTGGATGGCGTGGAGGTGCACAATGCCAAGACCAA |
| | | GCCCAGAGAGGAGCAGTACGCCTCTACATATAGGGTG |
| | | GTGAGCGTGCTGACCGTGCTGCACCAGGACTGGCTGAA |
| | | CGGCAAGGAGTATAAGTGCAAGGTGTCCAATAAGGCC |
| | | CTGCCCGCCCCCATCGAGAAGACAATCAGCAAGGCCA |
| | | AGGGCCAGCCTCGGGAGCCACAGGTGTACACCCTGCCT |
| | | CCATCCAGAGACGAGCTGACAAAGAACCAGGTGTCTC |
| | | TGACATGTCTGGTGAAGGGCTTCTATCCTAGCGATATC |
| | | GCCGTGGAGTGGGAGTCCAATGGCCAGCCAGAGAACA |
| | | ATTACAAGACCACACCCCCTGTGCTGGACTCCGATGGC |
| | | TCCTTCTTTCTGTATTCCAAGCTGACCGTGGATAAGTCT |
| | | CGGTGGCAGCAGGGCAACGTGTTCAGCTGTTCCGTGAT |
| | | GCACGAAGCCCTGCATAATCACTATACTCAGAAATCCC |
| | | TGTCCCTGTCACCTGGAAAGTGA |
| 63 | Nucleotide sequence | ATGGGCTGGTCTTGTATTATTCTGTTTCTGGTCGCAAC |
| | encoding VH7 | TGCTACAGGCGTGCATTCTCAAGTGCAGCTGCAGGAG |
| | | AGCGGACCTGGCCTGGTGAAGCCATCTCAGACCCTGT |
| | | CTCTCACATGTACCGTGTCTGGATATAGCATCACCAGC |
| | | GACTACGCCTGGAACTGGATCCGGCAGCACCCCGGCA |
| | | AGGGCCTGGAATGGATGGGCTACCTGTCCTACAGCGG |
| | | AGTTACATCTTATAATCCTAGCCTGAAAGGCAGAGTG |
| | | ACCATCAGCAGAGATACCTCCAAGAACCAGTTCAGCC |
| | | TGAAGCTGAGCAGCGTGACAGCCGCTGACACCGCCGT |
| | | GTACTACTGCGCCAGAAAGGGCACATACTACCGGTAC |
| | | GAGGGCAGCTACTGGTACTTCGACGTGTGGGGCCAGG |
| | | GCACCACCGTCACAGTGTCCAGCGCCAGCACCAAGGG |
| | | CCCTTCCGTGTTTCCCCTGGCCCCTTGCTCCCGGTCCAC |
| | | ATCTGAGAGCACCGCCGCCCTGGGCTGTCTGGTGAAG |
| | | GACTACTTCCCAGAGCCCGTGACCGTGAGCTGGAACA |
| | | GCGGCGCCCTGACAAGCGGCGTGCACACATTTCCCGC |
| | | CGTGCTGCAGAGCTCCGGCCTGTACTCCCTGTCTAGCG |
| | | TGGTGACAGTGCCTTCCTCTAGCCTGGGCACCAAGACA |
| | | TATACCTGTAACGTGGACCACAAGCCAAGCAATACCA |
| | | AGGTGGATAAGCGGGTGGAGTCTAAGTACGGCCCTCC |
| | | TTGCCCTCCATGTCCTGCTCCAGAGTTTCTGGGCGGCC |
| | | CTTCCGTGTTCCTGTTTCCACCCAAACCAAAGGACACA |
| | | CTGATGATCTCTAGAACACCAGAGGTGACCTGCGTGGT |
| | | GGTGGACGTGAGCCAGGAGGATCCCGAGGTGCAGTTC |
| | | AACTGGTACGTGGATGGCGTGGAGGTGCACAATGCCA |
| | | AGACCAAGCCAAGAGAGGAGCAGTTTAACTCTACATA |
| | | CAGGGTGGTGAGCGTGCTGACCGTGCTGCACCAGGAT |
| | | TGGCTCAACGGCAAGGAGTATAAGTGCAAGGTGTCCA |
| | | ATAAGGGCCTGCCCTCCTCTATCGAGAAGACAATCTCT |
| | | AAGGCTAAGGGCCAGCCAAGAGAGCCTCAGGTGTACA |
| | | CCCTGCCTCCAAGCCAGGAGGAGATGACAAAGAACCA |
| | | GGTGTCCCTGACATGTCTGGTGAAGGGCTTCTATCCCT |
| | | CCGACATCGCCGTGGAGTGGGAGTCTAATGGCCAGCC |
| | | TGAGAACAATTACAAGACCACACCCCCTGTGCTGGAC |
| | | TCTGATGGCAGCTTCTTTCTGTATTCCAGGCTGACCGT |
| | | GGATAAGTCTCGGTGGCAGGAGGGCAACGTGTTCAGC |
| | | TGCTCTGTGATGCACGAAGCCCTGCATAATCACTATA |
| | | CTCAGAAAAGTCTGTCACTGTCACTGGGAAAGTGA |
| 64 | Forward primer for | AGAGAAATACACGGAGCCCCA |
| | TRPM4 | |
| 65 | Reverse primer for | CCCACACTGGCACAAGGTC |
| | TRPM4 | |

What is claimed is:

1. A method of treating traumatic brain injury (TBI), comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

2. (canceled)

3. The method of claim 1, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14.

4. The method of claim 1, wherein the antibody or antigen-binding fragment thereof comprises:
a) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11;
b) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12;
c) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13;
d) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14;
e) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11; or
f) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

5. The method of claim 1, wherein the antibody or antigen-binding fragment thereof comprises
(a) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56; or
(b) a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

6. (canceled)

7. The method of claim 1, wherein the antibody:
(a) specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16; or
(b) inhibits TRPM4 activity; or
(c) inhibits TRPM4 current and/or internalizes membrane TRPM4 protein.

8. (canceled)

9. (canceled)

10. The method of claim 1, wherein the antibody or antigen-binding fragment thereof is encoded by a nucleic acid comprising a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63.

11. The method of claim 1, wherein the TBI is a result of one or more physical injury events, wherein the physical injury event is selected from the group consisting of collision, vehicle-related incident, fall, sport-related incident, military-related incident and explosion-related incident, wherein optionally the military-related incident is a military attack and wherein optionally the explosion-related incident is bomb explosion.

12. The method of claim 1, wherein the effective amount of the antibody or antigen-binding fragment thereof:
(a) is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg; or
(b) treats TBI by preventing damage to the blood-brain barrier and/or a neuron, and/or by alleviating the damage to the blood-brain barrier and/or a neuron; or
(c) treats TBI by reducing one or more symptoms of TBI in the subject, wherein optionally the symptom of TBI is selected from the group consisting of confusion, dizziness, sleep disturbances, breathing difficulties, anxiety, loss of consciousness, severe headache, repeated nausea and vomiting, slurred speech, walking difficulties, weakness in one side or part of the body, loss of sensory to one side or part of the body, vegetative state, and locked-in syndrome.

13. (canceled)

14. (canceled)

15. The method of claim 1, further comprising administering to the subject one or more interventions selected from the group consisting of:
(a) one or more surgical interventions; and
(b) one or more therapeutic agents.

16. The method of claim 1, further comprising:
(a) monitoring the subject by using a multimodality monitoring program; and/or
(b) providing neurocritical care to the subject.

17. The method of claim 15, wherein:
(a) the surgical intervention is selected from the group consisting of the removal of hematoma, mass lesion and/or cerebrospinal fluid, and bony decompression; or
(b) the therapeutic agent is selected from the group consisting of non-steroidal anti-inflammatory drugs, salicylates, corticosteroids, and mannitol.

18. (canceled)

19. A method of treating vascular dementia, comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

20. (canceled)

21. The method of claim 19, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14.

22. The method of claim 19, wherein the antibody or antigen-binding fragment thereof comprises:
a) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11;
b) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12;
c) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13;
d) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14;
e) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11; or
f) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

23. The method of claim 19, wherein the antibody or antigen-binding fragment thereof comprises:
(a) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56; or
(b) a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

24. (canceled)

25. The method of claim 19, wherein the antibody:
(a) specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16; or
(b) inhibits TRPM4 activity; or
(c) inhibits TRPM4 current and/or internalizes membrane TRPM4 protein.

26. (canceled)

27. (canceled)

28. The method of claim 19, wherein the antibody or antigen-binding fragment thereof is encoded by a nucleic acid comprising a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63.

29. The method of claim 19, wherein the vascular dementia is a result of one or more disorders selected from the group consisting of atherosclerosis, cerebral amyloid angiopathy (CAA), genetic small vessel angiopathy, inflammation, immune-mediated small vessel disease, venous collagenosis, and obesity.

30. The method of claim 19, wherein the effective amount of the antibody or antigen-binding fragment thereof:
(a) is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg; or
(b) treats vascular dementia by improving the cognitive function in the subject, wherein optionally the cognitive function is selected from the group consisting of memory, learning, thinking and perception; or
(c) treats vascular dementia by reducing one or more symptoms of vascular dementia in the subject, wherein optionally the symptom of vascular dementia is selected from the group consisting of confusion, dizziness, sleep disturbances, breathing difficulties, anxiety, difficulty in paying attention, difficulty in concentrating, difficulty in organization, reduced ability to organize thoughts or actions, memory impairment, restlessness, agitation, depression and apathy.

31. (canceled)

32. (canceled)

33. The method of claim 19, further comprising administering to the subject one or more interventions selected from the group consisting of:
(a) one or more surgical interventions; and
(b) one or more therapeutic agent.

34. The method of claim 33, wherein:
(a) the surgical intervention is selected from the group consisting of carotid endarterectomy (CEA) and carotid angioplasty and stenting (CAS); or
(b) the therapeutic agent is selected from the group consisting of antithrombotic drug, antihypertensive drug, antihyperlipidemic agent, and antidiabetic drug.

35. (canceled)


SEQUENCE LISTING (65 sequences)

SEQ ID NO: 1 - protein, 11 aa, synthetic construct
GYSITSDYAWN

SEQ ID NO: 2 - protein, 16 aa, synthetic construct
YLSYSGVTSYNPSLKG

SEQ ID NO: 3 - protein, 16 aa, synthetic construct
KGTYYRYEGSYWYFDV

SEQ ID NO: 4 - protein, 11 aa, synthetic construct
HANQNIDVWLS

SEQ ID NO: 5 - protein, 7 aa, synthetic construct
KTSNLHT

SEQ ID NO: 6 - protein, 9 aa, synthetic construct
QQGQSYPLT

SEQ ID NO: 7 - protein, 125 aa, synthetic construct
QVQLQESGPGLVKPSQTLSLTCTVSGYSITSDYAWNWIRQHPGKGLEWIGYLSYSGVTSYNPSLKGRVTISVDTSKNQFSLKLSSVTAADTAVYYCARKGTYYRYEGSYWYFDVWGQGTTVTVSS

SEQ ID NO: 8 - protein, 125 aa, synthetic construct
QVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSS

SEQ ID NO: 9 - protein, 125 aa, synthetic construct
QVQLQESGPGLVKPSQTLSLTCTVSGYSITSDYAWNWIRQHPGKGLQWMGYLSYSGVTSYNPSLKGRITISRDTSKNQFSLKLSSVTAADTAVYYCARKGTYYRYEGSYWYFDVWGQGTTVTVSS

SEQ ID NO: 10 - protein, 125 aa, synthetic construct
QVQLQESGPGLVKPSQTLSLTCTVSGYSITSDYAWNWIRQHPGKGLQWMGYLSYSGVTSYNPSLKGRITISRDTSKNQFFLKLSSVTAADTAVYYCARKGTYYRYEGSYWYFDVWGQGTTVTVSS

SEQ ID NO: 11 - protein, 107 aa, synthetic construct
DIQMTQSPSSLSASVGDRVTITCHANQNIDVWLSWYQQKPGKAPKLLIYKTSNLHTGVPSRFSGSGSGTDFTFTISSLQPEDIATYYCQQGQSYPLTFGGGTKLEIK

SEQ ID NO: 12 - protein, 107 aa, synthetic construct
DIQMTQSPSSLSASVGDRVTITCHANQNIDVWLSWYQQKPGKAPKLLIYKTSNLHTGVPSRFSGSGSGTDFTLTISSLQPEDIATYYCQQGQSYPLTFGGGTKLEIK

SEQ ID NO: 13 - protein, 107 aa, synthetic construct
DIQMTQSPSSLSASVGDRVTITCHANQNIDVWLSWYQQKPGNAPKLLIYKTSNLHTGVPSRFSGSGSGTDFTFTISSLQPEDIATYYCQQGQSYPLTFGGGTKLEIK

SEQ ID NO: 14 - protein, 107 aa, synthetic construct
DIQMTQSPSSLSASVGDRVTITCHANQNIDVWLSWYQQKPGNAPKLLIYKTSNLHTGVPSRFSGSGSGTDFTLTISSLQPEDIATYYCQQGQSYPLTFGGGTKLEIK

SEQ ID NO: 15 - protein, 21 aa, synthetic construct
RDSDSNCSSEPGFWAHPPGAQ

SEQ ID NO: 16 - protein, 4 aa, synthetic construct
EPGF

SEQ ID NO: 17 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagccttctcagaccctgagcctcacctgtaccgtgtccggatatagcatcacaagcgactacgcctggaattggatcagacagcacccaggcaagggcctggaatggatcggctacctgtcctacagcggcgtgacatcttataaccccagcctgaaaggaagagtgaccatcagcgtggacacctctaagaaccagttcagcctgaagctgtctagcgtcacagccgctgataccgccgtgtactactgcgccagaaagggcacctactaccggtacgagggcagctactggtacttcgacgtgtggggccagggcacaaccgtgaccgtttccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 18 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 19 - DNA, 1428 bp, synthetic construct
  source 1..1428: mol_type = other DNA
atgggctggtcatgcattattctgtttctggtcgcaactgctacaggcgtgcatagtcaagtgcagctgcaggagtctggacctggactggtgaagcctagccagaccctgagcctgacctgtaccgtctccggctacagcatcacctccgactacgcctggaattggatcagacagcaccccggcaagggcctccagtggatgggctacctgtcttattctggagtgacatcttacaacccaagcctgaaaggcagaatcaccatcagccgggacaccagcaagaaccagttcagcctgaagctgagcagcgtgaccgccgctgatacagccgtgtactactgcgccagaaagggcacctactaccggtacgaaggcagctattggtacttcgacgtgtggggccagggcacaacagtgaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 20 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagcctagccagaccctgtccctgacatgtaccgtgtctggctacagcatcacaagcgactacgcctggaattggatccggcagcacccaggaaagggcctccagtggatgggctatctgagctatagcggcgtgacatcctacaaccccagcctgaaaggaagaatcaccatcagcagagatacatctaagaaccagtttttcctgaagctgtctagcgtgaccgccgctgacaccgccgtgtactactgcgccagaaagggcacctactaccggtacgaaggcagctactggtacttcgacgtgtggggccagggcacaaccgtcaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 21 - DNA, 705 bp, synthetic construct
  source 1..705: mol_type = other DNA
atgggctggtcatgtattattctgtttctggtcgcaactgctacaggggtccatagtgatattcagatgacccagagccccagcagcctctccgccagcgtgggagatagagtgaccatcacctgtcacgccaaccagaatatcgacgtctggctgagctggtatcagcagaaacctggcaaggcccctaagctgctgatctacaagaccagcaacctgcacaccggcgtgccatctcggttcagcggctctggcagcggcaccgacttcacatttacaatcagctctctgcagcctgaggacatcgctacatactactgccagcaaggccagtcctaccccctgaccttcggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 22 - DNA, 705 bp, synthetic construct
  source 1..705: mol_type = other DNA
atgggctggtcatgtattattctgtttctggtcgcaactgctacaggggtccatagtgatattcagatgacccagtccccaagcagcctctccgccagcgtgggcgatagagtgaccatcacctgtcacgccaaccagaatatcgacgtgtggctgagctggtaccagcaaaagcccggcaaagcccctaagctgctgatctacaagaccagcaacctgcacaccggcgtccccagccggttcagcggatctggcagcggcaccgacttcaccctgacaatcagctctctgcagcctgaggacatcgctacatactactgccagcagggccagtcttatcctctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 23 - DNA, 705 bp, synthetic construct
  source 1..705: mol_type = other DNA
atgggctggtcatgtattattctgtttctggtcgcaactgctacaggggtccatagtgatattcagatgacccagagccccagcagcctgtccgccagcgtcggcgatagagtgacaatcacctgtcacgccaaccagaatatcgacgtgtggctgtcttggtatcagcaaaaacctggcaacgctcctaagctcctgatctacaagaccagcaacctgcacacaggcgtgccaagccggttcagcggcagcggatctggcaccgacttcaccttcacaatcagctctctgcagcctgaggacatcgccacctactactgccagcagggccagtcctaccccctgacctttggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 24 - DNA, 705 bp, synthetic construct
  source 1..705: mol_type = other DNA
atgggctggtcatgtattattctgtttctggtcgcaactgctacaggggtccatagtgatattcagatgacccagtctccatctagcctctccgccagcgtgggcgaccgggtgaccatcacctgtcacgccaaccagaacatcgacgtgtggctgagctggtatcagcagaaacctggaaatgcccctaagctgctgatctacaagaccagcaacctgcacaccggcgtccccagcagattcagcggcagcggctctggcaccgacttcaccctgacaatcagcagcctgcagcctgaggatatcgctacatactactgccagcaaggccagtcctaccccctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 25 - protein, 125 aa, synthetic construct
QVQLQESGPGLVKPSQSLSLTCTVTGYSITSDYAWNWIRQFPGNKLQWMGYLSYSGVTSYNPSLKGRISITRDTSKNQFFLQLSSVTPEDTATYYCARKGTYYRYEGSYWYFDVWGAGTTVTVSS

SEQ ID NO: 26 - protein, 107 aa, synthetic construct
DIQMNQSPSSLSASLGDTITITCHANQNIDVWLSWYQQKPGNVPKLLIYKTSNLHTGVPSRFSGSGSGTDFTLTISSLQPEDIATYYCQQGQSYPLTFGGGTKLEIK

SEQ ID NO: 27 - DNA, 1428 bp, synthetic construct
  source 1..1428: mol_type = other DNA
atgggctggtcatgcattattctgtttctggtcgcaactgctacaggcgtgcatagtcaggtgcagctgcaggagagcggacctggcctggtgaagcctagccagagcctgtccctcacctgtaccgtgaccggctactccatcacaagcgactacgcctggaattggatcagacagtttccaggcaacaagctgcagtggatgggctacctgtcttattctggcgtgacatcttacaaccccagcctgaaaggcagaatcagcatcacccgggacaccagcaagaaccagttcttcctgcaactgagcagcgtgacccctgaagataccgccacatactactgcgccagaaagggcacctactaccggtacgagggcagctattggtacttcgacgtgtggggcgctggaacaaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 28 - DNA, 705 bp, synthetic construct
  source 1..705: mol_type = other DNA
atgggctggtcatgtattattctgtttctggtcgcaactgctacaggggtccatagtgatatccagatgaaccagagcccttctagcctctccgccagcctgggcgacaccattacaatcacctgtcacgctaatcagaacatcgacgtgtggctgagctggtatcagcagaaacctggcaacgtgcccaagctgctgatctacaagaccagcaacctgcacaccggcgtgccatctagattcagcggatctggcagcggcaccgacttcaccctgaccatcagcagcctgcagcctgaggatatcgccacatactactgccagcaaggccagtcctaccccctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 29 - protein, 25 aa, synthetic construct
QVQLQESGPGLVKPSQSLSLTCTVT

SEQ ID NO: 30 - protein, 14 aa, synthetic construct
WIRQFPGNKLQWMG

SEQ ID NO: 31 - protein, 32 aa, synthetic construct
RISITRDTSKNQFFLQLSSVTPEDTATYYCAR

SEQ ID NO: 32 - protein, 11 aa, synthetic construct
WGAGTTVTVSS

SEQ ID NO: 33 - protein, 25 aa, synthetic construct
QVQLQESGPGLVKPSQTLSLTCTVS

SEQ ID NO: 34 - protein, 14 aa, synthetic construct
WIRQHPGKGLEWIG

SEQ ID NO: 35 - protein, 32 aa, synthetic construct
RVTISVDTSKNQFSLKLSSVTAADTAVYYCAR

SEQ ID NO: 36 - protein, 11 aa, synthetic construct
WGQGTTVTVSS

SEQ ID NO: 37 - protein, 14 aa, unidentified
WIRQHPGKGLEWMG

SEQ ID NO: 38 - protein, 32 aa, synthetic construct
RVTISRDTSKNQFSLKLSSVTAADTAVYYCAR

SEQ ID NO: 39 - protein, 14 aa, synthetic construct
WIRQHPGKGLQWMG

SEQ ID NO: 40 - protein, 32 aa, synthetic construct
RITISRDTSKNQFSLKLSSVTAADTAVYYCAR

SEQ ID NO: 41 - protein, 32 aa, synthetic construct
RITISRDTSKNQFFLKLSSVTAADTAVYYCAR

SEQ ID NO: 42 - protein, 23 aa, synthetic construct
DIQMNQSPSSLSASLGDTITITC

SEQ ID NO: 43 - protein, 15 aa, synthetic construct
WYQQKPGNVPKLLIY

SEQ ID NO: 44 - protein, 32 aa, synthetic construct
GVPSRFSGSGSGTDFTLTISSLQPEDIATYYC

SEQ ID NO: 45 - protein, 10 aa, synthetic construct
FGGGTKLEIK

SEQ ID NO: 46 - protein, 23 aa, synthetic construct
DIQMTQSPSSLSASVGDRVTITC

SEQ ID NO: 47 - protein, 15 aa, synthetic construct
WYQQKPGKAPKLLIY

SEQ ID NO: 48 - protein, 32 aa, synthetic construct
GVPSRFSGSGSGTDFTFTISSLQPEDIATYYC

SEQ ID NO: 49 - protein, 15 aa, synthetic construct
WYQQKPGNAPKLLIY

SEQ ID NO: 50 - protein, 474 aa, synthetic construct
MGWSCIILFLVATATGVHSQVQLQESGPGLVKPSQTLSLTCTVSGYSITSDYAWNWIRQHPGKGLEWIGYLSYSGVTSYNPSLKGRVTISVDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 474 aa, synthetic construct
MGWSCIILFLVATATGVHSQVQLQESGPGLVKPSQTLSLTCTVSGYSITSDYAWNWIRQHPGKGLQWMGYLSYSGVTSYNPSLKGRITISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 474 aa, synthetic construct
MGWSCIILFLVATATGVHSQVQLQESGPGLVKPSQTLSLTCTVSGYSITSDYAWNWIRQHPGKGLQWMGYLSYSGVTSYNPSLKGRITISRDTSKNQFFLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 471 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 233 aa, synthetic construct
MGWSCIILFLVATATGVHSDIQMTQSPSSLSASVGDRVTITCHANQNIDVWLSWYQQKPGKAPKLLIYKTSNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 233 aa, synthetic construct
MGWSCIILFLVATATGVHSDIQMTQSPSSLSASVGDRVTITCHANQNIDVWLSWYQQKPGKAPKLLIYKTSNLHTGVPSRFSGSGSGTDFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 233 aa, synthetic construct
MGWSCIILFLVATATGVHSDIQMTQSPSSLSASVGDRVTITCHANQNIDVWLSWYQQKPGNAPKLLIYKTSNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 233 aa, synthetic construct
MGWSCIILFLVATATGVHSDIQMTQSPSSLSASVGDRVTITCHANQNIDVWLSWYQQKPGNAPKLLIYKTSNLHTGVPSRFSGSGSGTDFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 1425 bp, synthetic construct
  source 1..1425: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcacaaagggccctagcgtgtttccactggccccctcctctaagtccacctctggaggaacagccgccctgggctgtctggtgaaggattatttcccagagcccgtgaccgtgtcctggaactctggcgccctgaccagcggagtgcacacatttccagccgtgctgcagagctccggactgtactccctgtctagcgtggtgaccgtgccttcctctagcctgggcacccagacatatatctgcaacgtgaatcacaagccctccaatacaaaggtggacaagaaggtggagcctaagtcttgtgataagacccacacatgccccccttgtcctgcaccagaggcagcaggaggaccttccgtgttcctgtttccacccaagccaaaggacaccctgatgatcagccgcacccctgaggtgacatgcgtggtggtggacgtgtcccacgaggatccagaggtgaagtttaactggtacgtggatggcgtggaggtgcacaatgccaagaccaagcctcgggaggagcagtacaactctacctatagagtggtgagcgtgctgacagtgctgcaccaggactggctgaacggcaaggagtataagtgcaaggtgtctaataaggccctgcccgcccctatcgagaagaccatcagcaaggcaaagggacagcctagggagccacaggtgtacacactgcctccatctagagacgagctgaccaagaaccaggtgagcctgacatgtctggtgaagggcttctatccaagcgatatcgccgtggagtgggagtccaatggccagcccgagaacaattacaagaccacaccccctgtgctggacagcgatggctccttctttctgtattccaagctgaccgtggataagtctcggtggcagcagggcaacgtgtttagctgttccgtgatgcacgaggccctgcacaatcactacacccagaagtctctgagcctgtcccccggcaagtga

SEQ ID NO: 62 - DNA, 1425 bp, synthetic construct
  source 1..1425: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacgcctctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 63 - DNA, 1416 bp, synthetic construct
  source 1..1416: mol_type = other DNA
atgggctggtcttgtattattctgtttctggtcgcaactgctacaggcgtgcattctcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaagtga

SEQ ID NO: 64 - DNA, 21 bp, synthetic construct
  source 1..21: mol_type = other DNA
agagaaatacacggagcccca

SEQ ID NO: 65 - DNA, 19 bp, synthetic construct
  source 1..19: mol_type = other DNA
cccacactggcacaaggtc